(19) United States
(12) Patent Application Publication
(10) Pub. No.: US 2005/0201986 A1
(43) Pub. Date: Sep. 15, 2005

(54) Cell targeting compositions and methods of using the same

(75) Inventors: David B. Weiner, Merion, PA (US); Jong J. Kim, North Wales, PA (US); Donghui Zhang, Philadelphia, PA (US)

Correspondence Address: COZEN O'CONNOR, P.C., 1900 MARKET STREET, PHILADELPHIA, PA, 19103-3508, US

(73) Assignee: The Trustees of the University of Pennsylvania

(21) Appl. No.: 11/123,600

(22) Filed: May 6, 2005

Related U.S. Application Data
(63) Continuation of application No. 09/680,690, filed on Oct. 6, 2000.
(60) Provisional application No. 60/157,871, filed on Oct. 6, 1999.

(51) Int. Cl. A61K 9/127; A61K 48/00; C12N 15/86

(52) U.S. Cl. 424/93.2; 435/456; 424/450

(57) ABSTRACT

Drug delivery compositions and methods of delivering compounds to specific cell types are disclosed. Vaccines and methods of immunizing individuals are disclosed. Compositions for drug delivery including gene therapy and methods of treating individuals using such compositions are disclosed.

### CROSS REFERENCE TO RELATED APPLICATIONS

This application claims priority to U.S. Provisional application number 60/157,871 which is incorporated herein by reference.

### FIELD OF THE INVENTION

The present invention relates to drug delivery compositions, to methods of delivering compounds to specific cell types, to improved vaccines, to methods of immunizing individuals, to compositions for drug delivery including gene therapy and to methods of treating individuals using such compositions.

### SUMMARY OF THE INVENTION AND PREFERRED EMBODIMENTS

On aspect of the present invention arises from the discovery that non-cellular particle that comprises the compound and a costimulatory ligand are particularly useful to deliver a compound into a cell that expresses costimulatory molecules. Accordingly, one aspect of the invention relates to methods of introducing a compound into cells that expresses costimulatory molecules. The methods comprise contacting the cell with a non-cellular particle that comprises the compound and a costimulatory ligand. In some embodiments, the compound is a nucleic acid molecule or protein. In some embodiments, the compound is DNA; in some embodiments, preferably plasmid DNA. In some embodiments the compound is DNA that comprises a nucleotide sequences that encodes a protein operably linked to regulatory elements functional in the cell. In some such embodiments the protein is an immunogenic protein, preferably in some embodiments, an immunogenic pathogen protein. Inother such embodiments, the compounds is DNA that comprises a nucleotide sequences that encodes an non-immunogenic protein operably linked to regulatory elements functional in the cell. In some embodiments, the compound is a viral protein. In some embodiments, the cell that expresses costimulatory molecules is a dendretic cell; in some embodiments, it is a macrophage cell. In some embodiments, the costimulatory ligand is an antibody or a native ligand of a costimulatory molecule. In some embodiments, the costimulatory ligand is a fusion protein that includes a costimulatory ligand portion and a viral protein portion. In some embodiments, the particle is selected from the group consisting of a viral particle, a protein complex, a liposome and a cationic amphiphile/DNA complex. In some embodiments, the particle is a non-replicating viral particle.

According to some aspects of the present invention, methods of introducing compounds into cells are provided which comprise contacting the cells with particles that comprises the compound and a fusion protein. The fusion protein comprises the extracellular region of CD28 and the transmembrane and cytoplasmic regions of HIV-1 gp41. The fusion protein provides an effective means to target the cell for delivery of the compound.

According to some aspects of the present invention, particles comprising a costimulatory ligand and a therapeutic protein or nucleic acid molecule that encodes a therapeutic protein are used to deliver therapeutic proteins to cells. The present invention provides methods of delivering therapeutic proteins to an individual comprising the step of administering to tissue of the individual at a site on said individual's body, a particle that comprises therapeutic protein or a nucleic acid molecule that encodes a therapeutic protein, and costimulatory ligand. In some embodiments, the therapeutic protein is a non-immunogenic therapeutic protein such as a growth factor or cytokine. The protein or DNA encoding the protein are provided as part of/within the particle. In some embodiments, DNA provided as part of/within the particle is plasmid DNA. In some embodiments, the particle is selected from the group consisting of a viral particle, a protein complex, a liposome and a cationic amphiphile/DNA complex. In some embodiments, the particle is a non-replicating viral particle.

Some embodiments of the invention provide methods of immunizing against cancer comprising administering to an individual, a cancer cell comprising a recombinant expression vector that encodes a costimulatory ligand. Some embodimebts of the invention relate to cancer cells that comprising a recombinant expression vector that encodes a costimulatory ligand.

According to some embodiments of the invention, a particle that comprises a compound and a costimulatory ligand is provided. In some embodiments, the costimulatory ligand is a fusion protein comprising the extracellular region of CD28 and the transmembrane and cytoplasmic regions of HIV-1 gp41. In some embodiments, the compound is a nucleic acid or protein. In some embodiments, the compound is DNA. In some embodiments, the compound is plasmid DNA. In some embodiments, the compound is DNA that comprises a nucleotide sequences that encodes a protein operably linked to regulatory elements functional in the cell. In some embodiments, the compound is DNA that comprises a nucleotide sequences that encodes an immunogenic protein operably linked to regulatory elements functional in the cell. In some embodiments, the compound is DNA that comprises a nucleotide sequences that encodes an immunogenic pathogen protein operably linked to regulatory elements functional in the cell. In some embodiments, the compound is DNA that comprises a nucleotide sequences that encodes an non-immunogenic protein operably linked to regulatory elements functional in the cell. In some embodiments, the particle is selected from the group consisting of a viral particle, a protein complex, a liposome and a cationic amphiphile/DNA complex. In some embodiments, the particle is a non-replicating viral particle.

A further aspect of the invention relates to methods of immunizing individuals. Such copmprise the steps of administering to tissue of the individual at a site on the individual's body, a DNA molecule that comprises a nucleotide sequence that encodes an immunogenic protein operably linked to regulatory elements. Subsequently, a particle that comprises an immunogenic protein is adminmstered to the individual. In some embodiments, the particle may further comprises a compound. In some embodiments, the compound may be a nucleic acid molecule. In some embodiments, the compound is DNA. In some embodiments, the compound is plasmid DNA. In some embodiments, the compound is DNA that comprises a nucleotide sequences that encodes an immunogenic protein operably linked to regulatory elements functional in the cell. In some embodiments, the compound is DNA that comprises a nucleotide sequences that encodes an immunogenic pathogen protein operably linked to regulatory elements functional in the cell. In some embodiments, the compound is DNA that comprises a nucleotide sequences that encodes an non-immunogenic protein operably linked to regulatory elements functional in the cell. In some embodiments, the particle is a viral particle. In some embodiments, the particle is a non-replicating viral particle. In some embodiments, the particle is a protein complex.

### DESCRIPTION OF PREFERRED EMBODIMENTS OF THE INVENTION

### DEFINITIONS

As used herein, the term “compound” is meant to refer to any molecule including, but not limited to, a nucleic acid molecule such as DNA or RNA, or a proteinaceous molecule such as a peptide, polypeptide or protein.

As used herein, the phrase “cell that expresses costimulatory molecules” is meant to refer to any cell that express one or more costimulatory molecules. Such cells are generally antigen presenting cells such as macrophage, granulocyte, dendretic, monocyte,or B cells. Examples of costimulatory molecules are CD80, CD86, CD40, ICOSL, ICAM-1, 41BB, M-CSFR, FLT3, CCR-5, CCR-3, and CCR-2.

As used herein, the term “non-cellular particle” is meant to refer to any particulate structure except a cell.

As used herein, the phrase “costimulatory ligand” is meant to refer to a molecule that specifically binds to a costimulatory molecule. The costimulatory ligand is a preferably protein, more preferably an anti-costimulatory molecule antibody, a natural ligand that is specific for the costimulatory molecule, fragments thereof or a fusion protein which includes a portion which specifically binds to a costimulatory molecule. In some embodiments, the portion of a fusion protein which specifically binds to a costimulatory molecule is an anti-costimulatory molecule antibody, a natural ligand that is specific for the costimulatory molecule, or fragments thereof. The fusion protein may further comprise portions which perform other functions.

As used herein, the term “antibody” is meant to refer to antibodies, as well as antibody fragments such as FAb and F(Ab)₂ fragments. Antibodies may, in some preferred embodiments, be monoclonal antibodies, primatized antibodies or humanized antibodies. Antibodies may, in some preferred embodiments, be murine or human antibodies.

As used herein, the term “natural ligand that is specific for the costimulatory molecule” is meant to refer to the cellular protein present on cells which binds to the costimulatory moleucle present on another cell. For example, CD28 and CTLA-4 are both natural ligands for CD80, CD28 is also a natural ligand for CD86, the natural ligand for CD40 is CD40L, the natural ligand for ICOSL is ICOS, the natural ligand for ICAM-1 is LFA-3, the natural ligand for 41BB is 41BBL, the natural ligand for MCSFR is MCSF, the natural ligand for FT3 is FL3L, the natural ligand for CCR2, CCR3 and CCR5 are MCP-3, and RANTES.

As used herein, the term “cationic amphiphile/DNA complex” is meant to refer to a complex arising from the mixture of DNA and one or more cationic amphiphiles.

As used herein the term “desired protein” is meant to refer to peptides and protein encoded by gene constructs of the present invention which either act as target proteins for an immune response or as a therapeutic or compensating protein in gene therapy regimens.

As used herein, the term “genetic therapeutic” refers to a pharmaceutical preparation that comprises a genetic construct that comprises a nucleotide sequence that encodes a therapeutic or compensating protein.

As used herein, the term “therapeutic protein” is meant to refer to proteins whose presence confers a therapeutic benefit to the individual.

As used herein, the term “compensating protein” is meant to refer to proteins whose presence compensates for the absence of a fully functioning endogenously produced protein due to an absent, defective, non-functioning or partially functioning endogenous gene.

Delivery of Compounds to Cells, Immunization and Delivery of Therapeutic Agents

Methods

The present invention relates to methods of introducing compounds into cells that express costimulatory molecules, and to non-cellular particles useful in such methods. According to the methods of the present invention, cells that express costimulatory molecules are contacted with non-cellular particles that comprise a compound in combination with a costimulatory ligand. The costimulatory ligand component of the particle specifically target the cells that express costimulatory molecules. The particles bind to the cells and are taken up by them, thus delivering the compound into the cell.

According to some aspects of the present invention, methods of immunizing individuals are provided. Such methods comprise the step of administering to tissue of the individual at a site on the individual's body, a non-cellular particle that comprises an immunogenic protein or a nucleic acid molecule that encodes an immunogenic protein. The particle additionally comprises costimulatory ligand. The particles bind to the cells and are taken them, thus delivering the immunogenic protein or a nucleic acid molecule that encodes an immunogenic protein into the cell. An immune response is generated against the immunogenic protein delivered to the cell or against the expression product of a nucleic acid molecule which encodes an immunogenic protein and which is taken up by and expressed in the cell.

The present invention may be used to immunize an individual against all pathogens such as viruses, prokaryote and pathogenic eukaryotic organisms such as unicellular pathogenic organisms and multicellular parasites.

Another aspect of the present invention provides a method of conferring a broad based protective immune response against hyperproliferating cells that are characteristic in hyperproliferative diseases and to a method of treating individuals suffering from hyperproliferative diseases. As used herein, the term “hyperproliferative diseases” is meant to refer to those diseases and disorders characterized by hyperproliferation of cells. Examples of hyperproliferative diseases include all forms of cancer and psoriasis. The present invention provides a method of treating individuals suffering from hyperproliferative diseases. In such methods, the compound provides a target protein against which an immune response that will be specific for proteins expressed by hyperproliferating cells. While the present invention may be used to immunize an individual against one or more of several forms of cancer, the present invention is particularly useful to prophylactically immunize an individual who is predisposed to develop a particular cancer or who has had cancer and is therefore susceptible to a relapse. Developments in genetics and technology as well as epidemiology allow for the determination of probability and risk assessment for the development of cancer in individual. Using genetic screening and/or family health histories, it is possible to predict the probability a particular individual has for developing any one of several types of cancer. Similarly, those individuals who have already developed cancer and who have been treated to remove the cancer or are otherwise in remission are particularly susceptible to relapse and reoccurrence. As part of a treatment regimen, such individuals can be immunized against the cancer that they have been diagnosed as having had in order to combat a recurrence. Thus, once it is known that an individual has had a type of cancer and is at risk of a relapse, they can be immunized in order to prepare their immune system to combat any future appearance of the cancer.

The present invention provides a method of treating individuals suffering from autoimmune diseases and disorders by conferring a broad based protective immune response against targets that are associated with autoimmunity including cell receptors and cells which produce “self”-directed antibodies.

According to some aspects of the present invention, methods of delivering therapeutic compounds to individuals are provided. According to such methods, the compound is a therapeutic compound. In some embodiments, the compound is therapeutic protein or a nucleic acid molecule that encodes a therapeutic protein. The methods comprise the step of administering to tissue of the individual at a site on the individual's body, a non-cellular particle that comprises an therapeutic protein or a nucleic acid molecule that encodes an therapeutic protein. The particle additionally comprises costimulatory ligand. The particles bind to the cells and are taken them, thus delivering the therapeutic protein or a nucleic acid molecule that encodes an therapeutic protein into the cell. The therapeutic protein is thus delivered directly to the cell or is produced in the cell by the of the nucleic acid molecule which encodes it and is taken up in the cell.

Some aspects of the present invention relate to gene therapy; that is, to compositions for and methods of introducing nucleic acid molecules into the cells of an individual exogenous copies of genes which either correspond to defective, missing, non-functioning or partially functioning genes in the individual or which encode therapeutic proteins, i.e. proteins whose presence in the individual will eliminate a deficiency in the individual and/or whose presence will provide a therapeutic effect on the individual thereby providing a means of delivering the protein by an alternative means from protein administration.

Compounds

Compounds which can be delivered to cells by the methods of the invention may be any molecule. In some embodiments, the compound is a nucleic acid molecule such as DNA or RNA. In some embodiments, the compound is a proteinaceous molecule such as a peptide, polypeptide or protein.

In some embodiments, the compound is a protein molecule. In some embodiments, the compound is an immunogenic protein. In some embodiments, the compound is a non-immunogenic protein molecule.

Examples of immunogenic proteins includes pathogen antigens, proteinaceous allergans, immunogenic proteins associated with cancer cells, and immunogenic proteins associated with cells involved in autoimmune diseases.

Pathogen antigens may be derived from all pathogens such as viruses, prokaryote and pathogenic eukaryotic organisms such as unicellular pathogenic organisms and multicellular parasites. The present invention is particularly useful to immunize an individual against those pathogens which infect cells and which are not encapsulated such as viruses, and prokaryote such as gonorrhea, listeria and shigella. In addition, the present invention is also useful to immunize an individual against protozoan pathogens which include a stage in the life cycle where they are intracellular pathogens. As used herein, the term “intracellular pathogen” is meant to refer to a virus or pathogenic organism that, at least part of its reproductive or life cycle, exists within a host cell and therein produces or causes to be produced, pathogen proteins. Table 1 provides a listing of some of the viral families and genera for which vaccines according to the present invention can be made. DNA constructs that comprise DNA sequences which encode the peptides that comprise at least an epitope identical or substantially similar to an epitope displayed on a pathogen antigen such as those antigens listed on the tables are useful in vaccines. Moreover, the present invention is also useful to immunize an individual against other pathogens including prokaryotic and eukaryotic protozoan pathogens as well as multicellular parasites such as those listed on Table 2. Tables 1 and 2 include lists of some of the pathogenic agents and organisms for which genetic vaccines can be prepared to protect an individual from infection by them. In some preferred embodiments, the methods of immunizing an individual against a pathogen are directed against HIV, HTLV or HBV.

As used herein, the term “hyperproliferative-associated protein” is meant to refer to proteins that are associated with a hyperproliferative disease. To immunize against hyperproliferative diseases, a “hyperproliferative-associated protein” or a genetic construct that includes a nucleotide sequence which encodes a protein that is associated with a hyperproliferative disease is included ars the compound in the particle administered to an individual. In order for the hyperproliferative-associated protein to be an effective immunogenic target, it must be a protein that is produced exclusively or at higher levels in hyperproliferative cells as compared to normal cells. Target antigens include such proteins, fragments thereof and peptides which comprise at least an epitope found on such proteins. In some cases, a hyperproliferative-associated protein is the product of a mutation of a gene that encodes a protein. The mutated gene encodes a protein which is nearly identical to the normal protein except it has a slightly different amino acid sequence which results in a different epitope not found on the normal protein. Such target proteins include those which are proteins encoded by oncogenes such as myb, myc, fyn, and the translocation gene bcr/abl, ras, src, P53, neu, trk and EGRF. In addition to oncogene products as target antigens, target proteins for anti-cancer treatments and protective regimens include variable regions of antibodies made by B cell lymphomas and variable regions of T cell receptors of T cell lymphomas which, in some embodiments, are also used target antigens for autoimmune disease. Other tumor-associated proteins can be used as target proteins such as proteins which are found at higher levels in tumor cells including the protein recognized by monoclonal antibody 17-1A and folate binding proteins.

T cell mediated autoimmune diseases include Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors that bind to endogenous antigens and initiate the inflammatory cascade associated with autoimmune diseases. Vaccination against the variable region of the T cells would elicit an immune response including CTLs to eliminate those T cells.

In RA, several specific variable regions of T cell receptors (TCRs) which are involved in the disease have been characterized. These TCRs include Vβ-3, Vβ-14, Vβ-17 and Vα-17. Thus, vaccination with a particle that contains as the compound one of these proteins or a DNA construct that encodes at least one of these proteins will result in the generation of an immune response that will target T cells involved in RA. See: Howell, M. D., et al., 1991 Proc. Natl. Acad Sci. USA 88:10921-10925; Paliard, X., et al., 1991 Science 253:325-329; Williams, W. V., et al., 1992 J. Clin. Invest. 90:326-333; each of which is incorporated herein by reference.

In MS, several specific variable regions of TCRs which are involved in the disease have been characterized. These TCRs include Vβ-7 and Vα-10. Thus, vaccination with a particle that contains as the compound one of these proteins or a DNA construct that encodes at least one of these proteins will result in the generation of an immune response that will target T cells involved in MS. See: Wucherpfennig, K. W., et al., 1990 Science 248:1016-1019; Oksenberg, J. R., et al., 1990 Nature 345:344-346; each of which is incorporated herein by reference.

In scleroderma, several specific variable regions of TCRs which are involved in the disease have been characterized. These TCRs include Vβ-6, Vβ-8, Vβ-14 and Vα-16, Vα-3C, Vα-7, Vα-14, Vα-15, Vα-16, Vα-28 and Vα-12. Thus, vaccination with a particle that contains as the compound one of these proteins or a DNA construct that encodes at least one of these proteins will result in the generation of an immune response that will target T cells involved in scleroderma.

In order to treat patients suffering from a T cell mediated autoimmune disease, particularly those for which the variable region of the TCR has yet to be characterized, a synovial biopsy can be performed. Samples of the T cells present can be taken and the variable region of those TCRs identified using standard techniques. Particles useful to immunize against the disease can be prepared using this information.

B cell mediated autoimmune diseases include Lupus (SLE), Grave's disease, myasthenia gravis, autoimmune hemolytic anemia, autoimmune thrombocytopenia, asthma, cryoglobulinemia, primary biliary sclerosis and pernicious anemia. Each of these diseases is characterized by antibodies which bind to endogenous antigens and initiate the inflammatory cascade associated with autoimmune diseases. Vaccination against the variable region of antibodies would elicit an immune response including CTLs to eliminate those B cells that produce the antibody.

In order to treat patients suffering from a B cell mediated autoimmune disease, the variable region of the antibodies involved in the autoimmune activity must be identified. A biopsy can be performed and samples of the antibodies present at a site of inflammation can be taken. The variable region of those antibodies can be identified using standard techniques. Particles usedul to immunize against such diseases can be prepared using this information.

In the case of SLE, one antigen is believed to be DNA. Thus, in patients to be immunized against SLE, their sera can be screened for anti-DNA antibodies and a vaccine can be prepared which includes the variable region of those antibodies or DNA constructs that encode the variable region of such anti-DNA antibodies found in the sera.

Common structural features among the variable regions of both TCRs and antibodies are well known. The DNA sequence encoding a particular TCR or antibody can generally be found following well known methods such as those described in Kabat, et al. 1987 Sequence of Proteins of Immunological Interest U.S. Department of Health and Human Services, Bethesda Md., which is incorporated herein by reference. In addition, a general method for cloning functional variable regions from antibodies can be found in Chaudhary, V. K., et al., 1990 Proc. Natl. Acad. Sci. USA 87:1066, which is incorporated herein by reference.

In some embodiments the compound in the particle is a non-immunogenic protein which may serve as replacement protein in individuals suffering from diseases associated with defective, missing or non-functioning genes. The non-immunogenic proteins may alternatively be therapeutic proteins. In some embodiments the compound in the particle is a nucleic acid molecule which serves as: 1) replacement copies of defective, missing or non-functioning genes; 2) genetic templates for therapeutic proteins; 3) genetic templates for antisense molecules; or 4) genetic templates for ribozymes. In the case of nucleic acid molecules which encode proteins, the nucleic acid molecules preferably comprise the necessary regulatory sequences for transcription and translation in the cells of the animal. In the case of nucleic acid molecules which serve as templates for antisense molecules and ribozymes, such nucleic acid molecules are preferably linked to regulatory elements necessary for production of sufficient copies of the antisense and ribozyme molecules encoded thereby respectively. The nucleic acid molecules are free from retroviral particles and preferably provided as DNA in the form of plasmids.

In some of the embodiments of the invention that relate to gene therapy, the gene constructs contain either compensating genes or genes that encode therapeutic proteins. Examples of compensating genes include a gene which encodes dystrophin or a functional fragment, a gene to compensate for the defective gene in patients suffering from cystic fibrosis, an insulin, a gene to compensate for the defective gene in patients suffering from ADA, and a gene encoding Factor VIII. Additionally, genetic constructs which encode antibodies, such as single chain antibody components which specifically bind to toxic substances, can be administered. In some embodiments, antibvodies expressed in such cells can be secreted. In some preferred embodiments, the dystrophin gene is provided as part of a mini-gene and used to treat individuals suffering from muscular dystrophy. In some preferred embodiments, a mini-gene which contains coding sequence for a partial dystrophin protein is provided. Dystrophin abnormalities are responsible for both the milder Becker's Muscular Dystrophy (BMD) and the severe Duchenne's Muscular Dystrophy (DMD). In BMD dystrophin is made, but it is abnormal in either size and/or amount. The patient is mild to moderately weak. In DMD no protein is made and the patient is chair-bound by age 13 and usually dies by age 20. In some patients, particularly those suffering from BMD, partial dystrophin protein produced by expression of a mini-gene delivered according to the present invention can provide improved muscle function.

Examples of therapeutic proteins include the proteins themselves and the genes which encodes active proteins such as cytokines, growth factors, chemokines as well as toxins. In some embodiments, the protein is erythropoietin, interferon, LDL receptor, GM-CSF, IL-2, IL-4 or TNF. Therapeutic proteins or nucleic acid molecules that encode therapeutic proteins may be included in particles as a compound to be delivered to cells. Therapeutic proteins that are toxins cor otherwise toxic or cytostatic to the cellare useful for example when delivered to antigen presenting cells in patients with lymphoproliferative diseases. In addition to toxins, other anti-prolifertive proteins are antibodies, HIV Vpr and TGFβ. Therapeutic proteins that expand APC numbers include growth factors such as EPO, CSF and GCSF. Proteins which modulate immune responses may be delivered to cells in this manner in order to modulate immune responses in an individual.

Antisense molecules and ribozymes may also be delivered to the cells of an individual by introducing genetic material which acts as a template for copies of such active agents. These agents inactivate or otherwise interfere with the expression of genes that encode proteins whose presence is undesirable. Constructs which contain sequences that encode antisense molecules can be used to inhibit or prevent production of proteins within cells. Thus, production proteins such as oncogene products can be eliminated or reduced. Similarly, ribozymes can disrupt gene expression by selectively destroying messenger RNA before it is translated into protein. in some embodiments, cells are treated according to the invention using constructs that encode antisense or ribozymes as part of a therapeutic regimen which involves administration of other therapeutics and procedures. Gene constructs encoding antisense molecules and ribozymes use similar vectors as those which are used when protein production is desired except that the coding sequence does not contain a start codon to initiate translation of RNA into protein.

Ribozymes are catalytic RNAs which are capable of self-cleavage or cleavage of another RNA molecule. Several different types of ribozymes, such as hammerhead, hairpin, Tetrahymena group I intron, axhead, and RNase P are known in the art. (S. Edgington, Biotechnology 1992 10,256-262.) Hammerhead ribozymes have a catalytic site which has been mapped to a core of less than 40 nucleotides. Several ribozymes in plant viroids and satellite RNAs share a common secondary structure and certain conserved nucleotides. Although these ribozymes naturally serve as their own substrate, the enzyme domain can be targeted to another RNA substrate through base-pairing with sequences flanking the conserved cleavage site. This ability to custom design ribozymes has allowed them to be used for sequence-specific RNA cleavage (G. Paolella et al., EMBO 1992, 1913-1919.) It will therefore be within the scope of one skilled in the art to use different catalytic sequences from various types of ribozymes, such as the hammerhead catalytic sequence and design them in the manner disclosed herein. Ribozymes can be designed against a variety of targets including pathogen nucleotide sequences and oncogenic sequences. Certain preferred embodiments of the invention include sufficient complementarity to specifically target the abl-bcr fusion transcript while maintaining efficiency of the cleavage reaction.

Peptides, polypeptides and protein may be isolated from natural sources, synthesized or produced by recombinant methodology.

Recombinant expression vectors that comprises a nucleotide sequence that encodes proteins of the invention can be produced routinely. As used herein, the term “recombinant expression vector” is meant to refer to a plasmid, phage, viral particle or other vector which, when introduced into an appropriate host, contains the necessary genetic elements to direct expression of a coding sequence. One having ordinary skill in the art can isolate or synthesize a nucleic acid molecule that encodes a protein of the invention and insert it into an expression vector using standard techniques and readily available starting materials. The coding sequence is operably linked to the necessary regulatory sequences. Expression vectors are well known and readily available. Examples of expression vectors include plasmids, phages, viral vectors and other nucleic acid molecules or nucleic acid molecule containing vehicles useful to transform host cells and facilitate expression of coding sequences. The recombinant expression vectors of the invention are useful for transforming hosts.

Host cells that comprise the recombinant expression vector can be used to produce the protein. Host cells for use in well known recombinant expression systems for production of proteins are well known and readily available. Examples of host cells include bacteria cells such as E. coli, yeast cells such as S. cerevisiae, insect cells such as S. frugiperda, non-human mammalian tissue culture cells chinese hamster ovary (CHO) cells and human tissue culture cells such as HeLa cells.

In some embodiments, for example, one having ordinary skill in the art can, using well known techniques, insert DNA molecules into a commercially available expression vector for use in well known expression systems. For example, the commercially available plasmid pSE420 (Invitrogen, San Diego, Calif.) may be used for production of a CD80ΔC mutant protein in E. coli. The commercially available plasmid pYES2 (Invitrogen, San Diego, Calif.) may, for example, be used for production in S. cerevisiae strains of yeast. The commercially available MAXBAC™ complete baculovirus expression system (Invitrogen, San Diego, Calif.) may, for example, be used for production in insect cells. The commercially available plasmid pcDNA I or pcDNA3 (Invitrogen, San Diego, Calif.) may, for example, be used for production in mammalian cells such as Chinese Hamster Ovary cells. One having ordinary skill in the art can use these commercial expression vectors and systems or others to produce proteins of the invention using routine techniques and readily available starting materials. (See e.g., Sambrook et al., Molecular Cloning a Laboratory Manual, Second Ed. Cold Spring Harbor Press (1989) which is incorporated herein by reference.) Thus, the desired proteins can be prepared in both prokaryotic and eukaryotic systems, resulting in a spectrum of processed forms of the protein.

One having ordinary skill in the art may use other commercially available expression vectors and systems or produce vectors using well known methods and readily available starting materials. Expression systems containing the requisite control sequences, such as promoters and polyadenylation signals, and preferably enhancers, are readily available and known in the art for a variety of hosts. See e.g., Sambrook et al., Molecular Cloning a Laboratory Manual, Second Ed. Cold Spring Harbor Press (1989).

The expression vector including the DNA that encodes a protein is used to transform the compatible host which is then cultured and maintained under conditions wherein expression of the foreign DNA takes place. The protein of the invention thus produced is recovered from the culture, either by lysing the cells or from the culture medium as appropriate and known to those in the art. One having ordinary skill in the art can, using well known techniques, isolate the protein of the invention that is produced using such expression systems. The methods of purifying proteins of the invention from natural sources using antibodies which specifically bind to such protein are routine as is the methods of generating such antibodies (See: Harlow, E. and Lane, E., Antibodies: A Laboratory Manual, 1988, Cold Spring Harbor Laboratory Press which is incorporated herein by reference.). Such antibodies may be used to purifying proteins produced by recombinant DNA methodology or natural sources.

Examples of genetic constructs include coding sequences which encode a protein of the invention and which are operably linked to a promoter that is functional in the cell line into which the constructs are transfected. Examples of constitutive promoters include promoters from cytomegalovirus or SV40. Examples of inducible promoters include mouse mammary leukemia virus or metallothionein promoters. Those having ordinary skill in the art can readily produce genetic constructs useful for transfecting with cells with DNA that encodes proteins of the invention from readily available starting materials. Such gene constructs are useful for the production of proteins of the invention.

In addition to producing proteins of the invention by recombinant techniques, automated peptide synthesizers may also be employed to produce proteins of the invention. Such techniques are well known to those having ordinary skill in the art and are useful if derivatives which have substitutions not provided for in DNA-encoded protein production.

The proteins of the invention may be prepared by any of the following known techniques. Conveniently, the proteins of the invention may be prepared using the solid-phase synthetic technique initially described by Merrifield, in J. Am. Chem. Soc., 15:2149-2154 (1963) which is incorporated herein by reference. Other protein synthesis techniques may be found, for example, in M. Bodanszky et al., (1976) Peptide Synthesis, John Wiley & Sons, 2d Ed. which is incorporated herein by reference; Kent and Clark-Lewis in Synthetic Peptides in Biology and Medicine, p. 295-358, eds. Alitalo, K., et al. Science Publishers, (Amsterdam, 1985) which is incorporated herein by reference; as well as other reference works known to those skilled in the art. A summary of synthesis techniques may be found in J. Stuart and J. D. Young, Solid Phase Peptide Synthelia, Pierce Chemical Company, Rockford, Ill. (1984) which is incorporated herein by reference. Synthesis by solution methods may also be used, as described in The Proteins, Vol. II, 3d Ed., p. 105-237, Neurath, H. et al., Eds., Academic Press, New York, N.Y. (1976) which is incorporated herein by reference. Appropriate protective groups for use in such syntheses will be found in the above texts, as well as in J. F. W. McOmie, Protective Groups in Organic Chemistry, Plenum Press, New York, N.Y. (1973) which is incorporated herein by reference.

In general, these synthetic methods involve the sequential addition of one or more amino acid residues or suitable protected amino acid residues to a growing peptide chain. Normally, either the amino or carboxyl group of the first amino acid residue is protected by a suitable, selectively-removable protecting group. A different, selectively removable protecting group is utilized for amino acids containing a reactive side group, such as lysine.

Using a solid phase synthesis as an example, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to provide the final peptide. The peptide of the invention are preferably devoid of benzylated or methylbenzylated amino acids. Such protecting group moieties may be used in the course of synthesis, but they are removed before the peptides are used. Additional reactions may be necessary, as described elsewhere, to form intramolecular linkages to restrain conformation.

In some embodiments, proteins may be produced in transgenic animals. Transgenic non-human mammals useful to produce recombinant proteins are well known as are the expression vectors necessary and the techniques for generating transgenic animals. Generally, the transgenic animal comprises a recombinant expression vector in which the nucleotide sequence that encodes a protein is operably linked to a mammary cell specific promoter whereby the coding sequence is only expressed in mammary cells and the recombinant protein so expressed is recovered from the animal's milk. One having ordinary skill in the art using standard techniques, such as those taught in U.S. Pat. No. 4,873,191 issued Oct. 10, 1989 to Wagner and U.S. Pat. No. 4,736,866 issued Apr. 12, 1988 to Leder, both of which are incorporated herein by reference, can produce transgenic animals which produce a desired protein. Preferred animals are goats, and rodents, particularly rats and mice.

In some embodiments, the compound is a nucleic molecule, preferably a DNA molecule. In some embodiments, the nucleic acid molecule is an antisense molecule, which when taken up by the cell, prevents or otherwise inhibits expression of a gene in the cell. In some embodiments, the nucleic acid molecule is a gene construct which contains a coding sequence operably linked to regulatory elements necessary for gene expression of a nucleic acid molecule in the cell.

In addition to a coding sequence, the elements of a gene construct include a promoter, an initiation codon, a stop codon, and a polyadenylation signal. In addition, enhancers are often required for gene expression of the sequence that encodes the protein. It is necessary that these elements be operable linked to the sequence that encodes the desired proteins and that the regulatory elements are operably in the individual to whom they are administered.

Initiation codons and stop codon are generally considered to be part of a nucleotide sequence that encodes the desired protein. However, it is necessary that these elements are functional in the individual to whom the gene construct is administered. The initiation and termination codons must be in frame with the coding sequence.

Promoters and polyadenylation signals used must be functional within the cells of the individual.

Examples of promoters useful to practice the present invention, especially in the production of a genetic vaccine for humans, include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metalothionein.

Examples of polyadenylation signals useful to practice the present invention, especially in the production of a genetic vaccine for humans, include but are not limited to human and bovine growth hormone polyadenylation signals, SV40 polyadenylation signals and LTR polyadenylation signals. In particular, the SV40 polyadenylation signal which is in pCEP4 plasmid (Invitrogen, San Diego Calif.), referred to as the SV40 polyadenylation signal, is used.

In addition to the regulatory elements required for DNA expression, other elements may also be included in the DNA molecule. Such additional elements include enhancers. The enhancer may be selected from the group including but not limited to: human Actin, human Myosin, human Hemoglobin, human muscle creatine and viral enhancers such as those from CMV, RSV and EBV.

Genetic constructs of the invention can be provided with mammalian origin of replication in order to maintain the construct extrachromosomally and produce multiple copies of the construct in the cell. Plasmids pCEP4 and pREP4 from Invitrogen (San Diego, Calif.) contain the Epstein Barr virus origin of replication and nuclear antigen EBNA-1 coding region which produces high copy episomal replication without integration.

In some preferred embodiments related to immunization applications, nucleic acid molecule(s) are delivered which include nucleotide sequences that encode immunogenic proteins, and additionally, genes for proteins which further enhance the immune response against such target proteins. Examples of such genes are those which encode cytokines and lymphokines such as α-interferon, gamma-interferon, platelet derived growth factor (PDGF), GC-SF, GM-CSF, TNF, epidermal growth factor (EGF), IL- 1, IL-2, IL-4, IL-6, IL-8, IL- 10, IL-12 and B7.2.

In order to maximize protein production, regulatory sequences may be selected which are well suited for gene expression in the cells into which the construct is to be administered. Moreover, codons may be selected which are most efficiently transcribed in the cell. One having ordinary skill in the art can produce DNA constructs which are functional in the cells.

In some embodiments, the compound is a DNA molecule. In some embodiments, the compounds is a DNA molecule that is a plasmid. In some embodiments, the compound is a DNA molecule that comprises a nucleotide sequences that encodes a protein operably linked to regulatory elements functional in the cell. In some embodiments, the compound is a DNA molecule that comprises an immunogenic protein operably linked to regulatory elements functional in the cell. In some embodiments, the compound is a DNA molecule that comprises an immunogenic pathogen protein operably linked to regulatory elements functional in the cell. In some embodiments, the compound is a DNA molecule that comprises a non-immunogenic protein operably linked to regulatory elements functional in the cell.

DNA vaccines are described in U.S. Pat. No. 5,593,972, U.S. Pat. No. 5,589,466, PCT/US90/01515, PCT/US93/02338, PCT/US93/048131, and PCT/US94/00899, and the priority applications cited therein each of the patents and published patent applications, which are each incorporated herein by reference. In addition to the delivery protocols described in those applications, alternative methods of delivering DNA are described in U.S. Pat. Nos. 4,945,050 and 5,036,006, which are both incorporated herein by reference.

According to some embodiments, the compounds is a protein which includes viral sequences which function to package the compound in the viral particle. In some embodiments, the viral sequences are viral proteins. In some embodiments, the viral sequences are fragments of viral proteins which retain their ability to complex with other viral proteins in the assembly of viral particles. In some embodiments, the particle is an HIV particle and the compound is a fusion protein which includes sequences of the HIV Vpr protein. The fusion protein which includes sequences of the HIV Vpr protein are packaged in the HIV particle.

Non-Cellular Particles

The non-cellular particles according to these aspects of the invention include, but are not limited to, viral particles, protein complexes, liposomes and cationic amphiphile/DNA complexes. According to the invention, such non-cellular particles include a costimulatory molecule ligand or fusion protein which includes a costimulatory molecule ligand portion in order to target the particles to the cells which display costimulatory molecules which bind to the costimulatory molecule ligand or fusion protein displayed by the particle. It has been discovered that in addition to delivering the particles to the cells for localization to cells that display the costimulatory molecule, the particles according to the present invention which are delivered to and localized to cells that display the costimulatory molecule are taken up by the cells.

According to some embodiments of the invention, the particles are viral particles. In preferred embodiments, the particles are non-replicating viral particles. U.S. Pat. No. 5,714,316, which is incorporated herein by reference, describes the design and production of viral particles which display heterologous protein sequences on the viral particle envelope. The present invention provides an improvement to this technology by providing as the heterologous protein, either a costimulatory molecule ligand or fusion protein which includes a costimulatory molecule ligand portion. In some embodiments, the particles are HIV, HSV, HCV or Papillomavirus particles, preferably non-replicating.

Examples of viral particles according to the invention include non-replicating HIV particles, adenovirus particles, and adenovirus-like particles. Non-replicating viruses are produced using packaging cell lines. Packaging systems are described in each of the following U.S. patents which are incorporated herein by reference: U.S. Pat. Nos. 5,932,467, 5,952,225, 5,932,467, 5,928,913, 5,919,676, 5,912,338, 5,888,767, 5,872,005, 5,866,411, 5,843,723, 5,834,256, 5,753,500, 5,739,018, 5,736,387, 5,723,287, 5,716,832, 5,710,037, 5,693,531, 5,672,510, 5,665,577, 5,622,856, 5,587,308 and 5,585,254.

According to some embodiments, the particles are attenuated vaccines which are improved by providing them with costimulatory ligands to target cells that express costimulatory molecules. Any of the commercially available attenuated vaccines including those currently being investigated such as those undergoing preclinical or clinical premarket testing may be improved by the present invention.

According to some embodiments of the invention, the particles are liposome particles. U.S. Pat. Nos. 4,873,089, 5,227,470 and 5,258,499, which are incorporated herein by reference, describe methods of preparing liposomes that contain proteins displayed on their surfaces in order to target the liposomes to a cell with a cellular protein on its surface that specifically binds to the protein on the surface of the liposome. The present invention provides a specific application of this technology by providing as the receptor ligand, either a costimulatory molecule ligand or fusion protein which includes a costimulatory molecule ligand portion. Liposomes include positive charged, negative charged and neutral liposomes.

According to some embodiments of the invention, the particles are cationic amphiphile/DNA complexes. U.S. Pat. Nos. 5,837,533, 5,459,127 and Behr, J. P., et al. (1989) Proc. Natl. Acad. Sci. USA 86:6982-6986, which are each incorporated herein by reference, describe the design and production of receptor targeted cationic amphiphile/DNA complexes in which positively charged lipophilic compounds are provided with receptor ligands. The cationic amphiphilic compounds contain receptor ligand moieties which are displayed on the surface of complexes formed when the cationic amphiphile is mixed with DNA. Such teachings may also be applied to cationic lipid/DNA complexes such as those described in U.S. Pat. Nos. 5,955,365, 5,948,767, 5,945,400, 5,939,401, 5,935,936, 5,932,241, 5,925,628, 5,916,803, 5,910,488, 5,908,635, 5,891,468, 5,885,613, 5,830,430, 5,827,703, 5,783,565 and 5,767,099, which are incorporated herein by reference. In some embodiments, receptor ligand moieties are not linked to any molecule or are linked to neutral lipids which are mixed with the cationic amphiphile and DNA and incorporated into any complexes formed thereby. According to the present invention, cationic amphiphile/DNA are provided with receptor ligands that are costimulatory molecule ligands. Such complexes are targeted to cells that display costimulatory molecules. The complexes localize to and are taken up by the cells.

According to some embodiments of the invention, the particles are protein complexes which comprise two or more protein molecules. The protein complexes comprise a compound to be delivered and a costimulatory ligand.

Cells

The present invention provides methods of delivering compounds to a cells that expresses costimulatory molecules. Typically, cells that express costimulatory molecules are antigen presenting cells. In some embodiments, the method is directed at delivering compounds to a cell that expresses costimulatory molecules that is a dendretic cell. In some embodiments, the method is directed at delivering compounds to a cell that expresses costimulatory molecules that is a macrophage cell.

By delivering immunogens to these cells, immune responses can be generated. By delivering therapeutic proteins which modulate immune responses to these cells, immune responses can be modified. By delivering toxins to these cells, immune responses can be reduced. By delivering grwowth factors to these cells, immune responses can be enhanced.

Ligands

The costimulatory ligand is a molecule that specifically binds to a costimulatory molecule. In some embodiments, the costimulatory ligand is a protein, preferably an anti-costimulatory molecule antibody, a natural ligand that is specific for the costimulatory molecule or a fusion protein which comprises either an anti-costimulatory molecule antibody, natural ligand or functional fragment thereof.

Anti-costimulatory molecule antibody can be prepared from readily availalble starting materials using routine techniques. Antibodies against CD80, CD86, CD40, ICOSL, ICAM-1, 41BB, MCSFR, FLT3, CCR-5, CCR-3 an d CCR-2 may be used in particles of the invention in order to target the particles to cells expressing CD80, CD86, CD40, ICOSL, ICAM-1, 41BB, MCSFR, FLT3, CCR-5, CCR-3 and CCR-2 respectively.

Alternatively, natural ligands of CD80, CD86, CD40, ICOSL, ICAM-1, 41BB, MCSFR, FT3, CCR-5, CCR-3 and CCR-2 may be provided as costimulatory ligands in order to to target the particles to cells expressing CD80, CD86, CD40, ICOSL, ICAM-1, 41BB, MCSFR, FLT3, CCR-5, CCR-3 and CCR-2 respectively. The natural ligands include: CD28 and CTLA-4 which are both natural ligands for CD80; CD28, a natural ligand for CD86; CD40L, the natural ligand for CD40; ICOS, the natural ligand for ICOSL; LFA-3 the natural ligand for ICAM-1; 41BBL, the natural ligand for 41BB; MCSF, the natural ligand for MCSFR; FL3L, the natural ligand for FLT3; MCP3 and RANTES, the natural ligand for CCR-5, CCR-3 and CCR-2. The methods for preparing or otherwise obtaining these proteins are well known.

In some embodiments, the costimulatory ligand is a fusion protein which includes a costimulatory ligand portion. In some embodiments, the costimulatory ligand is portion is an anti-costimulatory molecule antibody. In some embodiments, the costimulatory ligand is portion is a complete natural costimulatory ligand molecule. In some embodiments, the costimulatory ligand portion is a fragment of a natural costimulatory ligand molecule which retains its ability to bind to a costimulatory molecule.

In some embodiments the costimulatory ligand is a fusion protein which comprises amino acid sequences which function in particle assembly or are involved in localizing the fusion protein on the particle. For example, in some embodiments the fusion protein further comprises viral protein sequences which function in particle assembly such that the fusion protein becomes part of a viral particle. In some embodiments, the costimulatory ligand is a fusion protein that includes a costimulatory ligand portion and a viral protein portion. In some embodiments, the viral protein portion is a complete viral protein molecule. In some embodiments, the viral protein portion is a fragment of a viral protein. In some embodiments, the viral protein portion is a fragment of a viral protein that comprise the internal domain and transmembrane regions of a viral protein linked to a functional costimulatory ligand portion. In some embodiments, the fusion protein consists of the portions of the viral protein which are responsible for viral entry inot the cell. In some embodiments, the fusion protein consists of the internal domain, transmembrane region and 5-20 amino acids of the external region of a viral protein linked to the extracellular region of a natural ligand of a costimulatory molecule.

In some embodiments, the viral protein portion is derived from a lentivirus such as HIV, from a flavivirus such as yellow fever virus, hepatitis C, JEV, West Nile River Virus or hepatitis E, from a pox virus such as avipox, fowlpox, vaccina, MVA or WR. In some embodiments, the viral protein portion is derived from influenza, rotavirus, cytomegalovirus, rabies virus. In some embodiments, the viral protein portion is selected from the group consisting of HIV gp41, HSV gD, HSV gC, HSV gI, HCV E1, Papillomavirus L1 and Papillomavirus L2. In some embodiments, the viral protein portion is selected from the group consisting of flavivirus E or M protein, poxvirus E or M protein, rotavirus G protein, rabies virus G protein, influenza virus HA portein and CMV GB protein. Importantly, the viral protein portion must contain sufficient viral sequences to be assembled within the viral particle when the particle is assembled. Viral sequences of the fusion protein interact with viral proteins to become included in the viral particle.

In some embodiments, the viral particle contains both a fusion protein and a wild type envelope protein. In some embodiments, the viral particle is free of wild type envelope protein.

In some embodiments, the fusion protein comprises two or more costimulatory ligand portions including two costimulatory ligand portions linked by a linker 15-30 amino acids, preferably about 22 amino acids. Such a fusion protein is particularly useful in preparing targeted liposomes. The duplicate costimulatory ligand portions may proceed N terminal to C terminal, linker, N terminal to C terminal which is particularly useful since it allows for the fusion protein to be prepare by recombinant means. In some embodiments, the formula is N terminal to C terminal, linker, C terminal to N terminal. In some embodiments, the formula is C terminal to N terminal, linker, N terminal to C terminal. In some embodiments, the formula is C terminal to N terminal, linker, C terminal to N terminal.

In some embodiments, the fusion protein comprises one or more costimulatory ligand portions linked to a hydrophobic tail.

In some embodiments, the fusion protein comprises one or more costimulatory ligand portions linked to a polycationic tail, such as a polylysine tail.

In some embodiments, the fusion protein comprises a costimulatory ligand portion linked to a second portion which complexes with a protein to be delivered. In such embodiments, the costimulatory ligand portion complexes to the compound directly.

Methodology and Compositions

Methods of the present invention comprise the step of administering non-cellular particles to tissue of the individual. In some preferred embodiments, the non-cellular particles are administered intramuscularly, intranasally, intraperatoneally, subcutaneously, intradermally, intravenously, by aerosol administration to lung tissue or topically or by lavage to mucosal tissue selected from the group consisting of vaginal, rectal, urethral, buccal and sublingual.

An aspect of the present invention relates to pharmaceutical compositions useful in the methods of the present invention. The pharmaceutical compositions comprise the non-cellular particles which comprise a compound and a costimulatory molecule or fusion protein. The pharmaceutical compositions further comprise a pharmaceutically acceptable carrier or diluent. The term “pharmaceutical” is well known and widely understood by those skilled in the art. As used herein, the terms “pharmaceutical compositions” and “injectable pharmaceutical compositions” are meant to have their ordinary meaning as understood by those skilled in the art. Pharmaceutical compositions are required to meet specific standards regarding sterility, pyrogens, particulate matter as well as isotonicity and pH. For example, injectable pharmaceuticals are sterile and pyrogen free.

In embodiments in which the pharmaceutical compositions according to the present invention comprise non-cellular particles which include nucleic acid molecules as the compound, a sufficient amount of non-cellular particles are adminstered to introduce about 1 ng to about 10,000 μg of nucleic acid to the tissue. In some preferred embodiments, the pharmaceutical compositions contain about 2000 μg, 3000 μg, 4000 μg or 5000 μg of DNA. In some preferred embodiments, the pharmaceutical compositions contain about 1000 μg of DNA. In some preferred embodiments, the pharmaceutical compositions contain about 10 ng to about 800 μg of DNA. In some preferred embodiments, the pharmaceutical compositions contain about 0.1 to about 500 μg of DNA. In some preferred embodiments, the pharmaceutical compositions contain about 1 to about 350 μg of DNA. In some preferred embodiments, the pharmaceutical compositions contain about 25 to about 250 μg of DNA. In some preferred embodiments, the pharmaceutical compositions contain about 100 μg DNA.

The pharmaceutical compositions according to the present invention are formulated according to the mode of administration to be used. One having ordinary skill in the art can readily formulate a vaccine or non-immunogenic therapeutic that comprises a genetic construct. In cases where intramuscular injection is the chosen mode of administration, an isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. The pharmaceutical preparations according to the present invention are provided sterile and pyrogen free. Pharmaceutical compositions according to the invention include delivery components in combination with nucleic acid molecules which further comprise a pharmaceutically acceptable carriers or vehicles, such as, for example, saline. Any medium may be used which allows for successful delivery of the nucleic acid. One skilled in the art would readily comprehend the multitude of pharmaceutically acceptable media that may be used in the present invention. Suitable pharmaceutical carriers are described in Remington's Pharmaceutical Sciences, A. Osol, a standard reference text in this field, which is incorporated herein by reference.

The pharmaceutical compositions of the present invention may be administered by any means that enables the active agent to reach the agent's site of action in the body of a mammal. The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Because peptides are subject to being digested when administered orally, oral formulations are formulated to enterically coat the active agent or otherwise protect it from degradation in the stomach (such as prenuetralization). Parenteral administration includes intravenous drip, subcutaneous, intraperitoneal or intramuscular injection, pulmonary administration, e.g., by inhalation or insufflation, or intrathecal or intraventricular administration. In preferred embodiments, parenteral administration, i.e., intravenous, subcutaneous, transdermal, intramuscular, is ordinarily used to optimize absorption. Intravenous administration may be accomplished with the aid of an infusion pump. The pharmaceutical compositions of the present invention may be formulated as an emulsion.

One skilled in the art would readily comprehend the multitude of pharmaceutically acceptable media that maybe used in the present invention. Suitable pharmaceutical carriers are described in Remington's Pharmaceutical Sciences, A. Osol, a standard reference text in this field, which is incorporated herein by reference. Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Compositions for oral administration include powders or granules, suspensions or solutions in water or non-aqueous media, capsules, sachets or tablets. Thickeners, flavoring agents, diluents, emulsifiers, dispersing aids or binders may be desirable. Compositions for parenteral, intravenous, intrathecal or intraventricular administration may include sterile aqueous solutions which may also contain buffers, diluents and other suitable additives and are preferably sterile and pyrogen free. Pharmaceutical compositions which are suitable for intravenous administration according to the invention are sterile and pyrogen free. For parenteral administration, the peptides of the invention can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. For example, a parenteral composition suitable for administration by injection is prepared by dissolving 1.5% by weight of active ingredient in 0.9% sodium chloride solution

The pharmaceutical compositions according to the present invention may be administered as a single dose or in multiple doses. The pharmaceutical compositions of the present invention may be administered either as individual therapeutic agents or in combination with other therapeutic agents. The treatments of the present invention may be combined with conventional therapies, which may be administered sequentially or simultaneously.

Dosage varies depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Formulation of therapeutic compositions and their subsequent administration is believed to be within the skill of those in the art. Usually, the dosage of peptide can be about 1 to 3000 milligrams per 50 kilograms of body weight; preferably 10 to 1000 milligrams per 50 kilograms of body weight; more preferably 25 to 800 milligrams per 50 kilograms of body weight. Ordinarily 8 to 800 milligrams are administered to an individual per day in divided doses 1 to 6 times a day or in sustained release form is effective to obtain desired results.

Preferred Components

In some embodiments, the costimulatory ligand is a fusion protein comprising the extracellular portion of CD28 linked to a portion of HIV gp41. The HIV gp41 portion provides for the fusion protein to be packaged in an HIV particle, which is prefarbly a non-replicating particle. The CD28 extracellular portion targets the viral particle to cells that express CD80 and CD86. HIV viral particles localized to these cells take up the viral particles. In some embodiments, the viral particles are provided with fusion proteins that include Vpr sequences that provide for assembly into the viral particle. In some embodiments, the compound is a nucleic acid molecules.

II—Mucosal Targeting

Another aspect of the present invention relates to compositions and methods for targeting mucosal cells. The description above can be adapted for delivery of any fo the compounds descreibed above using any of the particles described above by substituting, in place of a costimulatory ligand, a ligand that binds to proteins specifically displayed by mucosal cells. Proteins specifically displayed by mucosal cells include PCAMs and P selectin. Ligands to PCAMs and P selectin, incuding fusion proteins that include ligand portions, can be included in particles as described above.

III—Particle Boost

Another aspect of the present invention relates to the use of the non-cellular particles as a vaccine boost. According to this aspect of the invention, subsequent to initial immunization of an individual against an immunogen, the individual is boosted with a non-cellular particle that comprises an immunogen included in the initial immunization. The non-cellular particle boost provides a particularly effective boost following initial immunization.

In some embodiments, the initial immunization is done using a DNA vaccine. In some embodiments, the initial immunization is done using a recombinant viral vaccine. In some embodiments, the initial immunization is done using a protein subunit vaccine. In some embodiments, the initial immunization is done using a an attenuated vaccine. In some embodiments, the initial immunization is done using a killed/inactivated vaccine.

In some embodiments, the particles are non-cellular particles that comprise a costimulatory ligand.

In some embodiments, the particles are non-cellular particles that comprise a protein immunogen previously delivered in the initial immunization.

In some embodiments, the particles are non-cellular particles that comprise a nucleic acid molecule that encodes a protein immunogen previously delivered in the initial immunization.

In some embodiments, the particles are non-cellular particles that comprise a costimulatory ligand and either a protein immunogen previously delivered in the initial immunization and/or a nucelci acid molecule that encodes a protein immunogen previously delivered in the initial immunization.

III—Recombinant Cell Based Cancer Vaccines

Another aspect of the present invention relates to the use of recombinant cancer cells as cancer vaccines. The use of recombinant cancer cells as cancer vaccines is described in U.S. Pat. No. 5,935,569, which is incorporated by references. According to this aspect of the invention, the recombinant gene expressed by the cancer cell is a costimulatory ligand. The cancer vaccine is an autologous cancer cell transfected with an expression vector that comprises a sequence encoding a costimulatory ligand. The cancer cells expressing the costimulatory ligand are targeted to cells that express costimulatory molecules and the immune response against the cancer cells in enhanced. In preferred embodiments, the recombinant expression vector that comprises a sequence encoding a costimulatory ligand is transfected into cancer cells ex vivo and the transfected cells are then restored to the patient.

In some embodiments, the transfected cancer cell is further provided with an expression vector that includes a nucleotide sequence that encodes a death domain receptor or death domain signal or a toxin. Death domain receptors include, but are not limited to; Apo-1 (Oehm et al., J. Biol. Chem., 1992, 267(15), 10709-15; Accession Number X63717); Fas (Itoh et al., Cell, 1991, 66(2), 233-43; Accession Number M67454); TNFR-1 (Nophar et al., EMBO J., 1990, 9(10), 3269-78; Accession Number M67454); p55 (Loetscher et al., Cell, 1990, 61, 351-359; Accession Numbers M58286, M33480); WSL-1 (Kitson et al., Nature, 1996, 384(6607), 372-5; Accession Number Y09392); DR3 (Chinnaiyan et al., Science,1996, 274(5829), 990-2; Accession Number U72763); TRAMP (Bodmer et al., Immunity, 1997, 6(1), 79-88; Accession Number U75381); Apo-3 (Marsters et al., Curr. Biol., 1996, 6(12), 1669-76; Accession Number U7461 1); AIR (Degli-Esposti et al., direct submission, Accession Number U78029); LARD (Screaton et al., Proc. Natl. Acad. Sci. USA, 1997, 94(9), 4615-19; Accession Number U94512); NGRF (Johnson et al., Cell, 1986, 47(4), 545-554; Accession Number M14764); DR4 (Pan et al., Science, 1997, 276(5309), 111-113; Accession Number U90875); DR5 (Sheridan et al., Science, 1997, 277(5327), 818-821; Accession Number AF012535); KILLER (Wu et al., Nature Genetics, in press; TRAIL-R2 (MacFarlane et al, J. Biol. Chem., 1997, in press; Accession Number AF020501); TRICK2 (Screaton et al., Curr. Biol., 1997, in press; Accession Number AF018657); DR6 (Pan et al., unpublished; Accession Number AF068868). Death signals, i.e. proteins that interact with the death domain receptors include, but are not limited to; FADD (Chinnaiyan et al., Cell, 1995, 81(4), 505-12; Accession Number U24231); FAP-1 (Sato et al., Science, 1995, 268 (5209), 411-15; Accession Number L34583); TRADD (Hsu et al., Cell, 1995, 81(4), 495-504; Accession Number L41690); RIP (Stanger et al., Cell, 1995, 81(4), 513-23; Accession Number U25994); and FLICE (Muzio et al., Cell, 1996, 85(6); 817-27; Accession Number U58143); RAIDD (Lennon et al., Genomics, 1996, 33(1),151-2; Accession Number U79115). Death signals also include ligands that bind death domain receptors and initiate apoptosis include, but are not limited to; FAS-L (Alderson et al., J. Exp. Med., 1995, 181(1), 71-7; Accession Number U08137), and TNF, and mediators that interact with death domain receptors include, but are not limited to; FADD (Chinnaiyan et al., Cell, 1995, 81(4), 505-12; Accession Number U24231); MORT1 (Boldin et al., J. Biol. Chem., 1995, 270(14), 7795-8; Accession Number X84709); CRADD (Ahmad et al., Cancer Res., 1997, 57(4), 615-9; Accession Number U84388); and MyD88 (Bonnert et al, FEBS Lett., 1997, 402(1), 81-4; Accession Number U84408). Toxins include proteins which kill cells. Toxins include but are not limited to insect and snake venoms, bacterial endotoxins such as Psuedomoneus endotoxin, double chain ribosome inactivating proteins such as ricin including single chain toxin, and gelonin.

The methods of the present invention are useful in the fields of both human and veterinary medicine. Accordingly, the present invention relates to genetic immunization of mammals, birds and fish. The methods of the present invention can be particularly useful for mammalian species including human, bovine, ovine, porcine, equine, canine and feline species.

The Examples set out below include representative examples of aspects of the present invention. The Examples are not meant to limit the scope of the invention but rather serve exemplary purposes. In addition, various aspects of the invention can be summarized by the following description. However, this description is not meant to limit the scope of the invention but rather to highlight various aspects of the invention. One having ordinary skill in the art can readily appreciate additional aspects and embodiments of the invention.

### EXAMPLES

### Example 1

The following sequences identified by accession number and references are incorporated herein by reference.

Macrophage Colony-Stimulating Factor

Accession No. AAA59572
Cerretti, D. P. et al.
Mol. Immunol. 25 (8), 761-770 (1988)
Accession No. AAB51235
Visvader, J. and Verma, I. M.
Mol. Cell. Biol. 9 (3) 1336-1341 (1989)
Accession No. P09603
Wong et al.
Science 235 (4795) 1504-1508 (1987)
Cerretti et al.
Mol. Immunol. 25 (8) 761-770 (1988)
Kawasaki et al.
Science 230 (4723) 291-296 (1985)
Chemokine (C-C motif) receptor 5
Accession No. 4502639
Raport, C. J. et al.
J. Biol. Chem. 271 (29), 17161-17166 (1996)
   Monocyte Chemoattractant Protein (MCP-3)
Accession No. CAA50407
Minty, A. et al.
Eur. Cytokine Netw. 4 (2), 99-110 (1993)
Accession No. AAC03538
   pFLT3
fins-related tyrosine kinase 3
Accession No. 4758396
Small, D. et al.
Proc. Natl. Acad. Sci. U.S.A. 91, 459-463 (1994)
Accession No. P36888
Small et al.
Proc. Natl. Acad. Sci. U.S.A. 91, 459-463 (1994)
   pFLT3LG
fins-related tyrosine kinase 3 ligand
Accession No. 4503751
   4-1BB
Accession No. AAA53133
Alderson, M. R. et al.
Eur. J. Immunol. 24 (9), 2219-2227 (1994)
   4-1BBL
Accession No. P41273
Alderson, M. R. et al.
Eur. J. Immunol. 24 (9) 2219-2227 (1994)
   RANTES
Accession No. BAA76939
Liu, H. et al.
PNAS U.S.A. 96 (8), 45814585 (1999)
Accession No. 1065018
   CCR1/MIP1R
Accession No. P32246
Neote, K. et al.
Cell 72 (3) 415-425 (1993)
Gao, J. L. et al.
J. Exp. Med. 177 (5) 1421-1427 (1993)
Nomura, H. et al.
Int. Immunol. 5 (10) 1239-1249 (1993)
   CCR5
Accession No. P56493
Kuhmann, S. E. et al.
J. Virol. 71 (11) 8642-8656 (1997)
Murayama, Y. et al.
   CCR2
Accession No. P41597
Charo, I. F. et al.
PNAS, U.S.A. 91 (7) 2752-2756 (1994)
Yamagami, S. et al.
Biochem. Biophys. Res. Commun. 202 (2) 1156-1162 (1994)
Wong, L. M. et al.
J. Biol. Chem. 272 (2) 1038-1045 (1997)
   CCR3
Accession No. P51677
Combadiere, C. et al.
J. Biol. Chem. 270 (28) 16491-16494 (1995)
Combadiere, C. et al.
J. Biol. Chem. 270 30235 (1995)
Dougherty, B. L. et al.
J. Exp. Med. 183 (5) 2349-2354 (1996)
   CD40 Ligand
Accession No. P29965
Graf, D. et al.
Eur. J. Immunol. 22 (12) 3191-3194 (1992)
Hollenbaugh, D. et al.
Embo. J. 11 (12) 4313-4321 (1992)
Spriggs, M. K. et al.
Cell 72 291-300 (1993)
Spriggs, M. K. et al.
J. Exp. Med. 176 (6) 1543-1550 (1992)
   Gauchat et al.
Febs. Lett. 315 (3) 259-266 (1993)
   CD86
Accession No. 5901920
Azuma et al.
Nature 366 (6450) 76-79 (1993)
Reeves et al.
Mamm. Genome 8 (8) 581-582 (1997)
   CD80
Accession No. 4885123
Selvakumar et al.
Immunogenetic 36 (3) 175-181 (1992)
Freeman et al.
Blood 79 (2) 489-494 (1992)
   CD40
Accession No. 4507581
Stamenkovic et al.
Embo. J. 8 (5) 1403-1410 (1989)
   LFA-3
Accession No. BAA05922
   ICAM1
Accession No. AAB51145
   CD28
Accession No. 5453611
Lee et al.
J. Immunol. 145 (1) 344-352 (1990)

### Example 2

### Multi-Component DNA Immunization can Modulate Immune Responses in Primates and Provide Significant Immunity Against Immunodeficiency Viral Challenge

Non-human primates represent the most relevant challenge models for HIV vaccine studies. Studies in rodents have established that DNA vaccine potency can be modulated by including genes encoding Th1 or Th2 cytokines as part of the vaccine. We sought to evaluate the immunomodulatory effects of such a strategy in rhesus macaques. DNA vaccines for HIV env/rev and SIV gag/pol alone were evaluated for their immunogenicity and compared to these vaccines which also included IL-2 or IFN-γ (Th1) or IL-4 (Th2) cytokine cDNA constructs. The cytokines dramatically enhanced seroconversion induced by the vaccines and appeared to modulate cellular responses as well, although more modestly. Vaccinated animals were challenged intravenously with SHIV IIIB. Half of the animals in the vaccine or vaccine plus Th1 cytokine groups exhibited protection from infection based on sensitive limiting dilution co-culture, demonstrating a dramatic effect on viral replication of the vaccines tested. The protected animals were reboosted with SIV DNA vaccines (SIV and cytokine constructs) and were rechallenged i.v. with pathogenic SIV_{mac239}. All vaccinated animals were negative for viral co-culture and antigenemia. In contrast, the control animals exhibited antigenemia by 2 weeks post challenge and exhibited greater than 10 logs of virus/10⁶ cells in limiting dilution co-culture. The control animal exhibited CD4 cell loss and developed SIV related wasting within 14 weeks of high viral burden and subsequently failed to thrive. Vaccinated animals were virus-negative and remained healthy. While exact correlates of protection could include cellular responses, neutralizing antibody responses do not appear to correlate with control of viral replication and infection in these studies. These studies establish that multi-component DNA vaccines can directly impact viral replication and disease in a highly pathogenic challenge system, thus potentially broadening our immunological weapons against HIV.

Introduction

Nucleic acid or DNA inoculation is an important vaccination technique which delivers DNA constructs encoding specific immunogens directly into the host¹⁻⁸. These expression cassettes transfect host cells, which become the in vivo protein source for the production of antigen. This antigen then is the focus of the resulting immune response. Nucleic acid immunization is being explored as an immunization strategy against a variety of infectious diseases including HIV¹⁻⁸. To support the ultimate use of this vaccine technology in humans, it may be important to translate the results originally observed in small animal systems to successes in primate models⁹.

Primates represent the most relevant challenge system for HIV vaccine evaluation. Specifically, there are currently three different primate models for HIV vaccine studies. They include the HIV challenge model in chimpanzees and the SIV and chimeric SIV/HIV-1 (SHIV-1) challenge models in macaques. Chimpanzees can be infected by HIV isolates from humans, however, they rarely develop disease. In contrast, the SIV challenge model uses the SIV virus which replicates to high levels and causes an HIV-like disease in macaques. In an effort to test HIV envelope immunogens in an animal model where challenge can cause disease, the SHIV viruses were constructed by replacing SIV envelope genes with specific HIV-1 envelope genes¹⁰. The SHIV viruses replicate in macaques and represent an infectious challenge model for HIV-1 envelope based vaccines, and certain SHIV strains such as SHIV 89.6P are pathogenic.

To date, the use of DNA vaccines to induce protective immunity in primates has had mixed results. Two out of two chimpanzees inoculated with constructs encoding for HIV envelope and gag/pol proteins from strain MN were protected from an i.v. challenge with a high dose (250 chimpanzee ID₅₀) of a heterologous stock of HIV-1 SF2¹¹. On the other hand, an env DNA constructs alone has demonstrated unclear utility in the SHIV model in macaques¹². Two out of two rhesus monkeys primed with large doses of HIV-1 gp120 DNA vaccine constructs and boosted with gp160 protein were protected from an i.v. challenge with 25 TCID₅₀ of SHIV-1 HXB2¹². However, protein vaccines alone can protect in this model in a type specific fashion and protection is based on the ability of protein to boost the type-specific neutralizing antibody response. Thus, the role of DNA vaccines alone is of uncertain value in this model.

The protective effects of DNA vaccine constructs in the SIV challenge model have been significantly less encouraging. Seven rhesus macaques were immunized with DNA vaccines encoding both envelope (four different plasmids) and gag (one plasmid) genes of SIV and were challenged with pathogenic SIV_{mac251} after their sixth immunization. Although vaccines induced positive responses, none of the vaccinated animals were protected from infection or disease¹³. In fact, this is not a limitation of DNA vaccines alone as the only vaccination strategy that has been shown convincingly to protect primates from a pathogenic SIV challenge to date is the live attenuated SIV vaccines¹⁴. It would be encouraging to obtain protection without using live, replicating HIV-1 or SIV with additional immunization strategies such as DNA vaccines. However, to date this has been an elusive goal.

We and others have been investigating the use of molecular adjuvants as a method of enhancing and modulating immune responses induced by DNA immunogens. Co-delivery of these molecular adjuvants consisting of expression plasmid encoding for immunologically relevant molecules, including costimulatory molecules (CD80 and CD86), proinflammatory cytokines (IL-1α, TNF-α, and TNF-β), Th1 cytokines (IL-2, IL-12, IL-15, and IL-18), Th2 cytokines (IL-4, IL-5 and IL-10), and GM-CSF with DNA vaccine constructs led to modulation of the magnitude and direction (humoral or cellular) of the immune responses induced in mice^{5, 15}-17. To date the ability of this strategy to manipulate responses in primates has not been reported.

In this pilot study, we investigated the enhancement of humoral and cellular immune responses by cytokine gene co-delivery in primates. We co-immunized rhesus macaques (Macaca mulatta) with expression plasmids encoding for Th1 (IL-2 and IFN-γ) or Th2-type (IL-4) cytokines along with the DNA vaccine constructs encoding for HIV env/rev (pCEnv) and SIV gag/pol (pCSGag/pol) proteins. The effects of this modulation on immune responses and protection in both the SHIV and SIV model systems were examined.

Modulation of Immune Responses in Mice

Cytokines play a critical regulatory and signaling role in the development of an immune response. Cytokines, which act on lymphocytes, are of special interest because of their role in regulating cells of the immune system. For instance, the presence of IL-2, IFN-γ, and IL-12 activates the Tₕ0 precursor cell to become a Tₕ1 inflammatory T cells¹⁸. On the other hand, the release of IL-4, IL-5, or IL-10 results in a Tₕ0 precursor becoming an armed Tₕ2 helper cell¹⁸. IL-2 is produced primarily by stimulated T cells and is critical for the proliferation and clonal expansion of antigen-specific T cells¹⁹. IL-4 is a prominent Th2 cytokine which plays an important role in the induction of humoral immune responses²⁰.

The effects of co-delivery of IL-2, IFN-γ, or IL-4 cytokine genes on DNA vaccine induced responses were analyzed. Antisera from immunized mice were collected and analyzed for specific antibody responses against HIV-1 gp120 protein by ELISA. Data was generated for the gp120-specific antibody titer from sera collected at weeks 0, 2, 4, 6 and 8 post-DNA immunization. At a 1:128 dilution, sera from the groups immunized with pCEnv+IL-2, pCEnv+IFN-γ, and pCEnv+IL-4 demonstrated antibody responses against gp120 protein which were significantly greater than that of the group immunized with pCEnv alone. A similar result was noted with the groups immunized with pCGag/pol. Lymphoproliferative responses of DNA-immunized mice were measured. IL-2 co-administration with HIV-1 immunogens (pCEnv or pCGag/pol) resulted in a dramatic level of antigen-specific T helper cell proliferative responses. Co-immunizations with IFN-γ and IL-4 cDNA also resulted in enhancement of T cell proliferative responses.

Co-Delivery of IL-2, IFN-γ, and IL-4 Expression Cassettes in Rhesus Monkeys

Important for the ultimate use of this vaccine technology in humans is that the results originally observed in mouse systems translate to primate models. Previously, it has been reported that primates may have a limited ability to produce DNA vaccine-encoded proteins through direct genetic inoculation into muscle²¹. More specifically, it has been reported that DNA immunizations alone in primates are not sufficient to generate high levels of antigen-specific antibody responses. For instance, IM immunization of an HIV-1 gp120 DNA vaccine construct using a large dose (2 mg of DNA given 8 times at 4-week intervals) in rhesus monkeys elicited only a low level of antigen-specific binding and no detectable neutralizing antibodies¹². These observations indicate reduced immunogenecity of DNA vaccines in non-human primates, potentially limiting their utility.

We investigated whether the enhancement of immune responses observed in mice with co-immunization with cytokine genes could also be achieved in rhesus monkeys. Four groups of two rhesus monkeys each were immunized with various DNA vaccine constructs. The first group was immunized with HIV env/rev (pCEnv) and SIV gag/pol (pCSGag/pol) constructs. The second group was immunized with pCEnv+pCSGag/pol+IL-2 constructs. The third and fourth groups were immunized with pCEnv+pCSGag/pol+IL-4 and pCEnv+pCSGag/pol+IFN-γ, respectively. These monkeys were immunized with 200 μg of each DNA at weeks 0, 6, and 12, and boosted with 500 μg of each DNA at weeks 28 and 49. These constructs were mixed prior to injection.

Modulation of Humoral Responses with Th1 or Th2 Cytokine Gene Co-Immunization

Both pre- and post-immunization serum samples from the immunized monkeys were collected, and binding reactivity to recombinant HIV-1 gp120 envelope and SIV p27 gag proteins was determined by ELISA. Monkeys immunized with pCEnv+pCSGag/pol without cytokine had minimal levels of anti-gp120 or anti-p27 antibodies at any time following immunization. However, a significant enhancement of the levels of anti-gp120 or anti-p27 antibodies was observed in the animals immunized with pCEnv+pCSGag/pol+IL-2. The magnitude of antibody response enhancement with IL-2 co-delivery in monkeys was dramatically greater than the results observed in mice (4-fold increase in end-point titer in mice compared to 20-fold increase in macaques). IL-4 co-immunization also positively modulated the antigen-specific antibody responses. The group immunized with pCEnv+pCSGag/pol+IL-4 developed a high level of anti-gp120 antibodies. On the other hand, the macaques immunized with pCEnv+pCSGag/pol+IFN-γ had a more moderate but still enhanced response against gp120 and p27 proteins.

Furthermore, these antigen-reactive sera from the immunized monkeys were positive by western blot analysis. The sera collected from monkeys at 36 weeks post-immunization were analyzed by Western Blot analysis. The western blot assay is a more stringent criteria than protein ELISA. The pre-immune sera of each monkey did not show reactivity. Moreover, the animals immunized with pCEnv+pCSGag/pol did not show any reaction to the western blot strips. In contrast, the group immunized with pCEnv+pCSGag/pol+IL-2 showed reactivity in western blot to both gp41 and p27 proteins. Moreover, IL-4 co-immunization resulted in a reactivity to gp160 protein while IFN-γ co-immunization resulted in a reactivity to gp41 and p18 proteins. The intensity of reactivity to specific bands were similar to reactivity of control SHIV infected sera lane. These results demonstrate that antigen-specific antibody responses can be engineered to more higher and presumably more desirable levels through the use of cytokine genetic adjuvants in rhesus monkeys.

We examined the ability of the antibodies from immunized monkeys to neutralize homologous HIV-1MN and heterologous HIV IIIB virus (Table 1). Although the IL-2 co-immunized group showed high level of serum antibody responses, the animals in the IL-4 co-immunized group showed greater levels of neutralizing antibodies against the homologous HIV-1 MN isolate. On the other hand, none of the serum antibodies were able to neutralize HIV IIIB virus. It is possible that further boosting of these animals might have broadened the humoral immune responses.

Modulation of Cellular Responses with Th1 or Th2 Cytokine Gene Co-Immunization

The importance of both HIV-specific CD4+ Th cell and CD8+ CTL responses in controlling viral load in HIV infected individuals is under extensive investigation²²⁻²⁸. The effect of DNA immunization on the antigen-specific Th cell proliferative and CTL responses in macaques were also examined. As shown in Table 2, induction of antigen-specific Th cell proliferative responses against gp120 and p27 proteins were observed. The groups immunized with pCEnv+pCSGag/pol constructs as well as those co-immunized with Th1 cytokines IL-2 and IFN-γ appeared to have more frequent and higher level of proliferative responses than the group co-immunized with Th2 cytokine IL-4. On the other hand, the effects of Th1 cytokine gene co-delivery (IL-2 and IFN-γ) on the Th1 shift in immune responses were less clear in these animals as compared to observations in the murine system. Moreover, the animals within each group had different Th proliferative response profiles. For instance, monkey #1 had α-env and gag/pol Th responses. In contrast, monkey #2 displayed one of the lowest level of Th proliferative responses over the same period.

We also evaluated CTL responses to the HIV Env or SIV gag/pol-expressing targets using immortalized autologous cell lines. As shown in Table 3, we did not observe specific CTL response above 10% lysis in the groups immunized with pCEnv+pCSGag/pol or pCEnv+pCSGag/pol+IL-4. On the other hand, the group immunized with pCEnv+pCSGag/pol+IL-2 had env-specific CTL lysis against env-expressing targets greater than 10% at 2 of 3 time points prior to SHIV challenge. Similarly, the monkeys immunized with pCEnv+pCSGag/pol+IFN-γ had env and gag/pol-specific CTL lysis greater than 10% at week 50.

We further examined the immunization-induced cellular immune responses by analyzing the levels of cytokines released from stimulated T lymphocytes isolated from each animal. Cytokines play a key role in directing and targeting immune cells during the development of the immune response. For instance, IFN-γ is produced by Th1 and CD8+ T cells and is intricately involved in the regulation or development of anti-viral T cell-mediated immune responses^{22, 29}. In contrast, IL-10 is produced by many cell types including putative Th2 lymphocytes and has been shown to be a potent Th2-type cytokine^{30, 31}. Thus, analysis of these cytokines secreted by stimulated T cells may be important in elucidating the extent of cell-mediated responses following immunization²⁴. We observed that the level of IFN-γ secreted was higher in the group immunized with pCEnv+pCSGag/pol than that of the control group. We also observed that co-administration with IFN-γ plasmid enhanced the level of IFN-γ production by the stimulated cells while co-injection with IL-4 plasmid reduced the level of IFN-γ. In contrast, co-immunization with IL-2 did not affect the level of IFN-γ production detected. On the other hand, the level of IL-10 produced by each group were similar.

Protection from SHIV IIIB Challenge

Following analysis of immune responses, the eight rhesus monkeys as well as two control monkeys were challenged by i.v. route with 10 animal infective doses (AID₅₀) of cell free SHIV IIIB at week 53 (four weeks after the last immunization). Animals were then bled at 2, 3, 4, weeks after the challenge and were assessed for protection from infection using a standard sensitive limiting dilution co-culture analysis¹⁴. As expected, both control monkeys were infected within two weeks of viral challenge. In addition, both animals co-immunized with Th2 cytokine IL-4 were infected (3 and 7.5 logs of virus/10⁶ cells at week 2 post-infection). In contrast, 50% of animals immunized with pCEnv+pCSGag/pol or those co-immunized with IL-2 or IFN-γ were able to control viral infection due to i.v. challenge and exhibited no detectable virus in the limiting dilution co-culture assay.

We also analyzed the cytokine expression profiles from individual animals prior to challenge. The stimulated T cells from the protected rhesus macaques produced higher levels of IFN-γ than the unprotected animals (both control or immunized). On the other hand, the level of IL-10 produced by either the protected or unprotected groups was similar.

Furthermore, we examined the expression profiles of β-chemokines (MIP-1α, MIP-1β, RANTES, and MCP-1) from immunized animals at week 53, just prior to challenge. The β-chemokines MIP-1α, MIP-1β, and RANTES are the major HIV suppressive factors produced by CD8⁺ T cells for macrophage-tropic, but not T cell tropic, viruses³²⁻³⁷. Although MCP-1 is not one of the HIV suppressive factors, it has been shown play a critical role in cellular immune expansion in the periphery³⁸. The stimulated T cells from the protected rhesus macaques produced significantly higher levels of MIP-1β and RANTES than the unprotected animals (with p values of 0.05 and 0.02, respectively). In contrast, the levels of MIP-1α and MCP-1 production by the two groups were not significantly different.

Analysis of DNA Immunization for a Pathogenic SIV Challenge

Following SHIV IIIB challenge and subsequent analysis, the three protected monkeys from SHIV IIIB challenge were boosted twice with additional DNA constructs. A combination of HIV-2 and SIV env was used based on previous results in mice⁶. Monkey #1 was boosted with SIV env (pCSEnv), SIV gag/pol (pCSGag/pol), and HIV-2 env (pCH2Env). Monkey #3 was boosted with SIV env (pCSEnv), SIV gag/pol (pCSGag/pol), HIV-2 env (pCH2Env), and IL-2. Similarly, Monkey #5 was boosted with SIV env (pCSEnv), SIV gag/pol (pCSGag/pol), HIV-2 env (pCH2Env), and IFN-γ. These monkeys were immunized with 1 mg of each DNA at weeks 81 and 85.

We examined the level of antigen-specific T helper cell proliferative responses against SIV gp130 and HIV-2 gp105 proteins (Table 4). After 2 booster immunizations (at weeks 81 and 85), the monkey immunized with the pCSEnv+pCSGag/pol+pCH2Env did not show positive proliferative responses. The animals co-immunized with IL-2 or IFN-γ had positive proliferative responses to gp130 and gp105 proteins.

At week 89, these animals as well as one control macaque were challenged by i.v. route with 10 AID₅₀ of pathogenic SIV_{mac239}. Following the challenge with SIV_{mac239}, the animals were bled 2 and 4 weeks prior to challenge and at 0, 1, 2, 3, 4, 8, and 12 weeks following challenge. The infection with SIV_{mac239} was assessed by both the plasma antigenemia assay and the limiting dilution co-culture analysis. Using a plasma antigenemia assay, we observed a high level of p27 antigen in the plasma of the control animal at 2 weeks post-challenge, while we did not detect any p27 antigen in the three vaccine recipients. These results were substantiated by the limiting dilution co-culture analysis. The control animal was infected as virus was readily detected within one week of challenge, and the viral load remained high throughout the analysis period (greater than 10 logs of virus/10⁶ cells) while 100% of the immunized macaques showed absence of viral load when assayed by the limiting co-culture method through 12 weeks post-challenge.

In our previous study of DNA vaccine challenge in chimpanzees, we observed that protected animals, in contrast to unprotected animals, exhibited no virus antigenemia, a lack of co-culture positive virus, and a lack of branched chain positive virus¹¹. However, an examination of samples using enhanced sensitivity DNA assay revealed transient low level detectability in this assay, suggesting that control of viral replication by DNA vaccines alone is not sterilizing in this system. Accordingly, we re-evaluated samples from protected animals using enhanced sensitivity PCR assay from SHIV as well as SIV challenges. The SHIV data was again similar to the observation in the chimpanzee system. Two out of three protected animals showed transient positive signals, indicating that protection is not sterilizing in these animals (Table 5). The follow-up analysis demonstrated no detectable virus by co-culture or PCR techniques. Results were surprisingly different in the SIV challenge. In contrast to the control animal which was infected and progressed to disease rapidly, the 3 protected animals have not demonstrated any virus by co-culture on the enhanced sensitivity technology. Thus, if infection did occur even transiently, it would be at an exceedingly low level.

As early as 14 weeks post-SIV challenge, the CD4 and CDw29 cell percentages on the control animal began to decline while the uninfected animals maintained normal levels of CD4 and CDw29 cell percentages (Table 6). By 18 weeks, the infected control animal exhibited several adverse clinical symptoms such as weight loss, lethargy, ruffled fur, and diarrhea, consistent with SIV induced disease. In fact, the health of the control animal continually deteriorate and the animals was euthanized by week 30 post-challenge. All vaccinated animals remained healthy.

Discussion

One of the major obstacles in the development of a vaccine against HIV-1 is uncertainty regarding the exact immune correlates of protection³⁹. In studies of long-term non-progressor groups of HIV-infected individuals, evidence supports the notion that correlates of protection against HIV-1 could be provided by humoral, cellular, or even both arms of the immune response^{40, 41}. High levels of type-specific neutralizing antibody have been observed in protected primates in some homologous challenge models⁴²⁻⁴⁵. Neutralizing antibodies are susceptible to viral deception through antigenic diversity of HIV-1 envelope, and the ability of neutralizing antibody to prevent viral pathogenesis is still under considerable investigation^{46, 47}.

One of the hallmarks of HIV-1 disease progression is the loss of cellular immune function, and the presence of strong cellular responses might correlate with control of viral replication. In cases of acute HIV-1 infection studied by several investigators, viral clearance was associated with specific CTL activity in each case^{25, 26}. In addition, a subset (7 of 20) of occupationally exposed health care workers who were not infected possessed transient HIV-1 specific CTL response⁴⁸. HIV-1-specific CTLs were also found in a number of chronically exposed sex workers in Gambia who continue to resist infection with HIV-1²⁷. In spite of these studies supporting the role of neutralizing antibodies and CTLs in conferring immunity to infection, some vaccinated primates exhibiting both neutralizing antibody and CTL responses were not protected from subsequent viral challenge in the pathogenic SIV model⁴⁹.

In the current study, we observed that antigen-specific humoral and cellular immune responses can be modulated by the co-delivery of the cytokine molecular adjuvants. First, we observed that antigen-specific antibody responses can be enhanced by the co-delivery of the genes for IL-2, IFN-γ, and IL-4. This was a significant finding for several reasons. The observation of humoral response enhancement effects of IL-2 and IL-4 DNA constructs in mice translated to positive modulatory effects in rhesus macaques. In fact, the magnitude of antibody response enhancement with IL-2 or IL-4 co-delivery in monkeys was even greater than the results observed in mice. Moreover, the sera from IL-4 co-immunized monkeys had the highest level of neutralizing activity to homologous HIV-1 MN. These results indicate that the use of molecular adjuvants (especially IL-2 or IL-4) to enhance the antibody responses could be important in disease models such as hepatitis B where the generation of antibodies are significant and sufficient to provide protective immunity. In the HIV challenge models in primates, however, we observed that the magnitude of antibody responses as well as the induction of significant neutralizing antibodies was not clearly correlated with the protective immunity in this study. Rather, the results from this study suggest that cellular immunity may be more important in protection against a SHIV challenge in rhesus monkeys.

In general, the antigen-specific cellular immune responses appeared to be enhanced by the co-delivery of the Th1, but not Th2-type cytokine molecular adjuvants. For instance, the rate of positive CTL response increased with Th1-type (IL-2 and IFN-γ) cytokine co-delivery (from 0/6 to 2/6). Furthermore, the co-immunization with IFN-γ expressing construct induced a greater level of IFN-γ production by stimulated T cells. On the other hand, the magnitude of the modulation of Th proliferative responses using this strategy was less convincing. In this regard, the Th responses observed with the env and gag/pol construct were similar to the cellular responses which were observed in any of the cytokine co-delivered animals during the initial set of immunization. These data support that this approach can have a profound effect on immune responses in primates. Moreover, other adjuvants, which particularly drive cellular responses should be examined. This point is the somewhat supported by the results from the IL-4 co-immunized group. The animals in this group had the overall lowest level of cellular responses and all animals had culturable virus. Based on the small group size, additional studies will be necessary to clarify this important issue.

The data in general support the importance of Th1 cytokines and chemokines in protective immunity. In fact, the level of IFN-γ detected from lymphocytes in the protected monkeys was significantly greater than those of the unprotected control animals or the unprotected vaccinated animals. The protected rhesus macaques also produced significantly higher levels of MIP-1β and RANTES than the unprotected animals. These results support that induction of these two β-chemokines through immunization could be important in relation to protection. However, additional studies will be necessary to distinguish the value of such mechanism as a marker of immune activation associated with better challenge outcome.

These studies extend and confirm the ability of a multicomponent DNA vaccine strategy to generate protective responses in primates¹¹. It is interesting to note that Th1 phenotype resulted in 50% overall protection in a SHIV model. If one includes animals #1 and 2 in this category as DNA constructs alone are Th1 biased, then 3 of 6 vaccine recipients in Th1 groups were protected. Monkey #1 had high level of α-env and gag/pol T cell proliferative responses while monkey #2 displayed lowest level of T lymphoproliferative responses over the period. It is also interesting that monkey #1 was protected, while #2 was not protected. The one Th2 biased group in the study, IL-4, has 0 of 2 animals exhibiting control of viral replication. Accordingly, one possible correlate appears to be driving responses towards a Th1 phenotype, a theory that has been under investigation previously⁵⁰. This study tested a low dose immunization protocol as compared to doses in other studies^{12, 13}. The total dose of each plasmid was 1.6 mg. It will be important to try slightly more aggressive doses and re-examine protection in a larger number of animals. The challenge results here in the Th1 groups suggest that combination vaccines encoding greater proportions of the HIV genome are likely to generate better challenge results, and suggest an important role for gag/pol immunogens in this protection.

The subsequent DNA immunization strategy can provide protective immunity from pathogenic SIV_{mac239} challenge in 3 out of 3 immunized rhesus macaques. Vaccines based on recombinant or subunit proteins, virus vectors, and prime boost strategies have not provided a consistent level of protection against pathogenic SIV_{mac239} or SIV_{mac251}. The approach which has provided the best protection against pathogenic SIV i.v. challenge is infection with the genetically attenuated SIV with the deletion of accessory genes, and improving the safety of such vaccine is clearly an important goal. In this pilot study, we found that the DNA vaccination scheme we employed provided protection from antigenemia, viral detection, and most importantly, disease and death all immunized monkeys, strongly supporting additional studies in this area. The results are important as they establish that neutralizing antibodies are not the only mechanism to achieve these important outcomes. However, these studies only support and do not clearly establish which cellular arms are responsible for viral control in these models. Rather, they indicate that driving responses towards a Th1 type phenotype could be of some importance.

Whether the protection from SIV challenge is entirely due to DNA vaccines alone or due to DNA and SHIV challenge should be further studied. It is important to consider the role of SHIV challenge as a boosting agent for gag-specific cellular responses in this study although follow-up immune responses were not necessarily supportive of such boosting. However, these results clearly demonstrate that protection from pathogenic challenge can be achieved in the absence of viral replication that reaches a threshold level of replication for effective vaccination, a worry for vaccine safety⁵¹. Furthermore, significant control over viral set point and prevention of CD4 loss, disease, and death can be achieved in multiple non-human primate models of HIV through immunization approaches. This is encouraging for further development of a prophylactic vaccine for HIV-1, as it implies that viral set point can be controlled with a combination of vaccination techniques which are conceptually simple to design and likely to be safe to administer. However, ultimately, the use of primate models to predict effectiveness in human population is of considerable debate.

Methods

DNA Plasmids. DNA vaccine constructs expressing HIV-1 envelope protein (pCEnv) and gag/pol protein (pCGag/Pol) as well as those expressing SIV envelope protein (pCSEnv) and gag/pol protein (pCSGag/Pol) and HIV-2 rod envelope (pCH2Env) were prepared as previously described^{6, 52, 53}. The cytokine genes were cloned into the pCDNA3 expression vector (Invitrogen, Inc., San Diego, Calif.) as previously described^{15, 16}.
Mouse studies. Six to 8 weeks old female BALB/c mice (Harlan Sprague Dawley, Inc., Indianapolis, Ind.) were housed at the University of Pennsylvania. Mouse injection and immunology protocols were conducted as previously described^{15, 16, 38}.
Macaques. Rhesus monkeys (Macaca mulatta) were individually housed at the Primedica Mason Labs (Worcester, Mass.). All animal care and use procedures conformed to the revised Public Health Service Policy on Humane Care and Use of Laboratory Animals. Animals were anesthetized with ketamine HCL for all technical procedures.
Immunization and challenge virus inoculation. Monkeys were immunized intramuscularly (IM) in the quadriceps with DNA preparations formulated in phosphate buffered saline (PBS) and 0.25% bupivacaine-HCl (Sigma, St. Louis, Mo.)^{15, 16} on multiple occasions. At week 53 of the study, all monkeys were challenge intravenously (i.v.) with 10 AID₅₀ of SHIV IIIB (provided by Yichen Lu, Virus Research Institute). At week 89, a subset of animals that were negative for virus recovery following SHIV challenge were challenged IV with 10 AID₅₀ of SIV_{mac239} (provided by Ronald C. Desrosiers, New England Regional Primate Research Center). Naive control animals were included at each challenge timepoint.
ELISA. Serum antibody reactivity to recombinant HIV-1 envelope and SIV gag/pol proteins were analyzed by ELISA as previously described¹¹. Briefly, recombinant HIV gp120 or SIV p27 protein (ImmunoDiagnostics, Inc., Bedford, Mass.) was resuspended in PBS to a concentration of 0.5 μg/ml. Fifty μl (25 ng) of the each protein preparation was incubated in each of the ELISA wells overnight at 4° C. Plates were then rinsed with washing buffer (0.45% NaCl in deionized water containing 0.05% Tween-20) and blocked with blocking buffer (5% non-fat dry milk in PBS with 1% BSA and 0.05% Tween-20) for two hours at 37° C. Serum samples were then diluted in dilution buffer (5% non-fat dry milk in PBS with 0.05% Tween-20) at the appropriate dilutions and incubated in duplicate or triplicate in recombinant protein coated wells for one hour at 37° C., washed and then incubated for one hour at 37° C. with a goat anti-human Ig-horseradish peroxidase conjugate (Sigma Chemical Co, St. Louis, Mo.) diluted in dilution buffer at the concentration suggested by the manufacturer. After extensive washing the plates were developed with 3,3′,5,5′-tetramethylbenzidine dihydrochloride (TMB) substrate (100 μg/ml), the reaction was stopped with 2N H₂SO₄ and color development was quantitated at 450 nm. BSA coated wells were used as negative binding control wells in these assays. Specific binding (absorbance at 450 nm) was calculated by subtracting A₄₅₀ values from sera samples bound to BSA (that is, control) from A₄₅₀ values from sera samples bound to gp120; that is, experimental wells (A₄₅₀experimental-A₄₅₀control).
Neutralization Assay. The ability of sera to neutralize viral infection in vitro was assessed according to described methods⁵⁴. Supernatant, 50 μl containing 100 TCID₅₀ of HIV-1 MN or IIIB strains were preincubated with 50 μl of serial dilutions of experimental or control monkey serum and added to 3×10⁴ MT-2 target cells (100 μl). The infection of cells was determined by the presence of p27 antigen after 48 hours of incubation.
T helper cell proliferation assay. Peripheral blood lymphocytes were prepared as previously described¹¹. The isolated cell suspensions were resuspended to a concentration of 5×10⁵ cells/ml in a media consisting of RPMI 1640 (Gibco-BRL, Grand Island, N.Y.) with 10% fetal calf serum (Gibco-BRL). A 100 μl aliquot containing 5×10⁵ cells was immediately added to each well of a 96-well microtiter round bottom plate. Recombinant p27 or gp120 protein at the final concentrations of 5 μg/ml and 1 μg/ml were added to wells in triplicate. The cells were incubated at 37° C. in 5% CO₂ for three days. One μCi of tritiated thymidine was added to each well and the cells were incubated for 12 to 18 hours at 37° C. The plates were harvested and the amount of incorporated tritiated thymidine was measured in a Beta Plate reader (Wallac, Turku, Finland). Stimulation Index was determined from the formula:
   Stimulation Index (SI)=(experimental count/spontaneous count)
   Spontaneous count wells (media only) include 10% fetal calf serum which serves as irrelevant protein control. To assure that cells were healthy, Concanavalin A (Sigma) was used as a polyclonal stimulator positive control.
Cytotoxic T Lymphocyte Assay. A standard 5 hour ⁵¹Cr release CTL assay was performed on PBMCs from the inoculated and control monkeys as previously described¹¹. Cells for in vitro stimulation of T cells were prepared by infecting autologously transformed B-lymphoblastoid cell lines (LCLs) with a recombinant vaccinia virus which expressed HIV-1 envelope (vMN462) or SIV gag proteins. Prior to use the infected cells were fixed with 0.1% glutaraldehyde and blocked with a 0.1 mM glutamine solution. The fixed cells were incubated with the isolated PBMCs (effectors) for stimulation in CTL stimulator media (RPMI 1640 (Gibco-BRL), 10% fetal calf serum (Gibco-BRL), and recombinant IL-2 (40 U/ml) (Intergen, Purchase, N.Y.)) for 3 weeks. The LCLs infected with specific recombinant vaccinia virus or control recombinant vaccinia virus expressing β-galactosidase (vSC8) were also used as target cells. Cells incubated with the control vaccinia were used as targets to provide background levels of lysis.
Cytokine expression analysis. Supernatants from effectors stimulated for CTL assay were collected and tested for cytokine profile using ELISA kits for IFN-γ and IL-10 (Biosource International, Inc., Camarillo, Calif.). Expression of MIP-1α, MIP-1β, RANTES, and MCP-1 were analyzed using ELISA kits (Intergen).
Cell associated virus load by limiting dilution co-culture. Viral load was determined by limiting dilution co-culture of isolated PBMC with CEMx174 target cells using a method previously described¹⁴. Twelve serial 1:3 dilutions of PBMC, beginning with 10⁶ cells, were co-cultured in duplicate with 10⁵ CEMx174 cells per well in 24-well plates. Supernatant samples were collected after 21 days of culture and stored frozen at −70° C. until analysis for p27 antigen with the Coulter p27 antigen assay kit.
Plasma Antigenemia. Plasma samples were analyzed two weeks after challenge with SIV mac 239 to determine plasma p27 levels. The assays were conducted using the Coulter p27 antigen kit.
Plasma RNA. Plasma samples from whole blood collected in sodium citrate were analyzed for SIV RNA copies per ml using the branched DNA assay (bDNA) developed by Chiron Corporation, Emeryville, Calif.
Flow cytometry. Whole blood collected in EDTA was analyzed for lymphocyte subsets CD4 (OKT4a (Ortho) and Anti-Leu 3a (Becton Dickinson)), CD8 (Anti-Leu 2a (Becton Dickinson)), and CDw29 (4B4) (Coulter Immunology) after red blood cell lysis using methods previously described¹⁴. Briefly, antibody (volume dependent upon antibody) was added to 100 μL of whole blood and incubated for 10 minutes in the dark. Lysing solution (Becton Dickinson) was added and the samples were incubated for 10 minutes at room temperature. Stained cells were fixed with 0.5% paraformaldehyde. Samples were analyzed on a Becton Dickinson FACScan cytometer.

### REFERENCES, WHICH ARE EACH INCORPORATED HEREIN BY REFERENCE

1. Wolff, J. A., R. W. Malone, P. Williams, W. Chong, G. Acsadi, A. Jani, P. L. Felgner. Direct gene transfer into mouse muscle in vivo. Science. 247, 1465-1468 (1990).
2. Tang, D., M. DeVit, S. Johnston. Genetic immunization is a simple method for eliciting an immune response. Nature. 356, 152-154 (1992).
3. Wang, B., K. E. Ugen, V. Srikantan, M. G. Agadjanyan, K. Dang, Y. Refaeli, A. Sato, J. Boyer, W. V. Williams, D. B. Weiner. Gene inoculation generates immune responses against human immunodeficiency virus type 1. Proc. Natl. Acad. Sci. USA. 90, 4156-4160 (1993).
4. Ulmer, J. B., J. Donnelly, S. E. Parker, G. H. Rhodes, P. L. Felgner, V. L. Dwarki, S. H. Gromkowski, R. Deck, C. M. DeVitt, A. Friedman, L. A. Hawe, K. R. Leander, D. Marinez, H. Perry, J. W. Shiver, D. Montgomery, M. A. Liu. Heterologous protection against influenza by injection of DNA encoding a viral protein. Science 259, 1745-1749 (1993).
5. Kim, J. J., M. L. Bagarazzi, N. Trivedi, Y. Hu, M. A. Chattergoon, K. Dang, S. Mahalingam, M. G. Agadjanyan, J. D. Boyer, B. Wang, D. B. Weiner. Engineering of In Vivo Immune Responses to DNA Immunization Via Co-Delivery of Costimulatory Molecule Genes. Nature Biot. 15, 641-645 (1997).
6. Agadjanyan, M. G., N. N. Trivedi, S. Kudchodkar, M. Bennett, W. Levine, A. Lin, J. Boyer, D. Levy, K. Ugen, J. J. Kim, D. B. Weiner. An HIV-2 DNA vaccine induces cross reactive immune responses against HIV-2 and SIV. AIDS and Human Retroviruses 13, 1561-1572 (1997).
7. Tascon, R. E., M. J. Colston, S. Ragno, E. Stavropoulos, D. Gregory, & Lowrie, D. B. Vaccination against tuberculosis by DNA injection. Nature Med. 2, 888-892 (1996).
8. Conry, R. M., G. Widera, A. F. LoBuglio, J. T. Fuller, S. E. Moore, D. L. Barlow, J. Turner, N.-S. Yang, D. T. Curiel. Selected strategies to augment polynucleotide immunization. Gene Therapy 3, 67-74 (1996).
9. Kim, J. J., D. B. Weiner. DNA/genetic vaccination for HIV. Springer Sem Immunopathol 19, 174-195 (1997).
10. Schultz, A. & Hu, S. Primate models for HIV vaccines. AIDS 7, 5161-5170 (1993).
11. Boyer, J. D., K. E. Ugen, B. Wang, M. G. Agadjanyan, L. Gilbert, M. Bagarazzi, M. Chattergoon, P. Frost, A. Javadian, W. V. Williams, Y. Refaeli, R. B. Ciccarelli, D. McCallus, L. Coney, D. B. Weiner. Protection of chimpanzees from high-dose heterologous HIV-1 challenge by DNA vaccination. Nature Med. 3, 526-532 (1997).
12. Letvin, N. L., D. C. Montefiori, Y. Yasutomi, H. C. Perry, M.-E. Davies, C. Lekutis, M. Alroy, D. C. Freed, C. I. Lord, L. K. Handt, M. A. Liu, J. W. Shiver. Potent, protective anti-HIV immune responses generated by bimodal HIV envelope DNA plus protein vaccination. Proc. Natl. Acad. Sci. USA. 94, 9378-9383 (1997).
13. Lu, S. et al. Simian Immunodeficiency Virus DNA Vaccine Trial in Macaques. Journal of Virology 70, 3978-3991 (1996).
14. Wyand, M. S., K. H. Manson, M. Garcia-Moll, D. Montefiori, R. C. Desrosiers. Vaccine protection by a triple deletion mutant of simian immunodeficiency virus. J. Virol. 70, 3724-3733 (1996).
15. Kim, J. J., V. Ayyvoo, M. L. Bagarazzi, M. A. Chattergoon, K. Dang, B. Wang, J. D. Boyer, D. B. Weiner. In vivo Engineering of a Cellular Immune Response by Co-administration of IL-12 Expression Vector with a DNA Immunogen. J. Immunol. 158, 816-826 (1997).
16. Kim, J. J., N. N. Trivedi, L. Nottingham, L. Morrison, A. Tsai, Y. Hu, S. Mahalingam, K. Dang, L. Ahn, N. K. Doyle, D. M. Wilson, M. A. Chattergoon, A. A. Chalian, J. D. Boyer, M. G. Agadjanyan, D. B. Weiner. Modulation of Amplitude and Direction of In Vivo Immune Responses By Co-Administration Of Cytokine Gene Expression Cassettes With DNA Immunogens. Eur. J. Immunol. 28, 1089-1103 (1998).
17. Sin, J. I., J. J. Kim, J. D. Boyer, C. Huggins, T. Higgins, D. B. Weiner. In Vivo Modulation of Immune Responses and Protective Immunity against Herpes Simplex Virus-2 Infection using cDNAs expressing Th1 and Th2 Type Cytokines in gD DNA Vaccination. J. Virol. In Press.(1998).
18. Arai, K. F. L., A. Miyajima, S. Miyatake, N. Arai, T. Yokota. Cytokines: coordinators of immune and inflammatory responses. [Review]. Annu. Rev. Biochem. 59,783-836 (1990).
19. Morgan, D. A., F. W. Ruscetti, R. Gallo. Selective in vitro growth of T lymphocytes from normal human bone marrows. Science 193, 1007-1008 (1976).
20. Seder, R. A., W. E. Paul. Acquisition of lymphokine-producing phenotype by CD4+ T cells. Annu. Rev. Immunol. 12, 635-673 (1994).
21. Jiao, S., Williams, P., Berg, R. K., Hodgeman, B. A., Liu, L., Repetto, G., Wolff, J. A. Direct gene transfer into non-human primate myofibers in vivo. Human Gene Therapy 3, 21-33 (1992).
22. Rosenberg, E. S., J. M. Billingsley, A. M. Caliendo, S. L. Boswell, P. E. Sax, S. A. Kalams, B. D. Walker. Vigorous HIV-1-specific CD4+ T cell responses associated with control of viremia. Science 278, 1447-1450 (1997).
23. Ogg, G. S., X. Jin, S. Bonhoeffer, P. R. Dunbar, M. A. Nowak, S. Monard, J. P. Segal, Y. Cao, S. L. Rowland-Jones, V. Cerundolo. A. Hurley, M. Markowitz, D. D. Ho, D. F. Nixon, A. J. McMichael. Quantitation of HIV-1-specific cytotoxic T lymphocytes and plasma load of viral RNA. Science 279, 2103-2106 (1998).
24. Lekutis, C., J. W. Shiver, M. A. Liu, N. L. Letvin. HIV-1 env DNA vaccine administered to rhesus monkeys elicits MHC class II-restricted CD4+ T helper cells that secrete IFN-gamma and TNF-alpha. J. Immunol. 158, 4471-4477 (1997).
25. Koup, R. A., J. T. Safrit, Y. Cao, C. A. Andrews, G. McLeod, W. Borkowsky, C. Farthing, D. D. Ho. Temporal association of cellular immune responses with the initial control of viremia in primary human immunodeficiency virus type 1 syndrome. J. Virol. 68, 4650 (1994).
26. Borrow, P., H. Lewicki, H. Hahn, G. M. Shaw, M. B. A. Oldstone. Virus-specific CD8+ cytotoxic T lymphocyte activity associated with control of viremia in primary human immunodeficiency virus type 1 infection. J. Virol. 68, 6103 (1994).
27. Rowland-Jones, S., Sutton J, Ariyoshi K, Dong T, Gotch F, McAdam S, Whitby D, Sabally S, Allimore A, Corrah T, Takiguchi M, McMichael A, Whittle H. HIV-specific T-cells in HIV-exposed but uninfected Gambian women. Nature Medicine 1, 59-64 (1995).
28. Pinto, L. A., J. Sullivan, J. A. Berzofsky, M. Clerici, H. A. Kessler, A. L. Landay, G. M. Shearer. Env-specific cytotoxic T lymphocyte responses in HIV seronegative health care workers occupationally exposed to HIV-contaminated body fluids. J. Clin. Invest. 96, 867-876 (1995).
29. Clerici, M., D. R. Lucey, J. A. Berzofsky, L. A. Pinto, T. A. Wynn, S. P. Blatt, M. J. Dolan, C. W. Hendrix, S. F. Wolf, G. M. Shearer. Restoration of HIV-specific cell-mediated immune responses by interleukin-12 in vitro. Science 262, 1721-1724 (1993).
30. Fiorentino, D. F., M. W. Bond, T. R. Mosmann. Two types of mouse T helper cell. IV. Th2 clones secrete a factor that inhibits cytokine production by Th1 clones. J. Exp. Med. 170, 2081-2095 (1989).
31. de Waal-Malefyt, R., J. Abrams, B. Bennett, C. G. Figdor, and J. de Vries. Interleukin 10 (IL-10) inhibits cytokine synthesis by human monocytes: an autoregulatory role of IL-10 produced by monocytes. J. Exp. Med. 174, 1209-1220 (1991).
32. Cocchi, F., A. L. DeVico, A. Garzino-Demo, S. K. Arya, R. C. Gallo, P. Lusso. Identification of RANTES, MIP-1α, and MIP-1b as the major HIV-suppressive factors produced by CD8+ T cells. Science 270, 1811-1815 (1995).
33. Dragic, T., V. Litvin, G. P. Allaway, S. R. Martin, Y. X. Huang, K. A. Nagashima, C. Cayanan, P. J. Maddon, R. A. Koup, J. P. Moore, W. A. Paxton. HIV-1 entry into CD4+ cells is mediated by the chemokine receptor CC-CKR-5. Nature 381, 667-673 (1996).
34. Deng, H. K., R. Liu, W. Ellemeier, S. Choe, D. Unutmaz, M. Burkhart, P. Di Marzio, S. Marmon, R. E. Sutton, C. M. Hill, C. B. Davis, S. C. Peiper, T. J. Schall, D. R. Littman, N. R. Landau. Identification of a major co-receptor for primary isolates of HIV-1. Nature 381, 661-666 (1996).
35. Choe, H., M. Farzan, Y. Sun, N. Sullivan, B. Rollins, P. D. Ponath, L. Wu, C. R. Mackay, G. LaRosa, W. Newman, N. Gerard, C. Gerard, J. Sodroski. The b-chemokine receptor CCR3 and CCR5 facilitate infection by primary HIV-1 isolates. Cell 85, 1135-1148 (1996).
36. Doranz, B. J., J. Rucker, Y. Yi, R. J. Smyth, M. Samson, S. C. Peiper, M. Parmentier, R. G. Collman, R. W. Doms. A dual-tropic primary HIV-1 isolate that uses fusin and the b-chemokine receptros CKR-5, CKR-3, and CKR-2b as fusion cofactors. Cell 85, 1149-1158 (1996).
37. Alkhatib, G., C. Combadiere, C. C. Boder, Y. Feng, P. E. Kennedy, P. M. Murphy, E. A. Berger. CC CKR5: A RANTES, MIP-1α, MIP-1b receptor as a fusion cofactor for macrophage-tropic HIV-1. Science 272, 1955-1958 (1996).
38. Kim, J. J., L. K. Nottingham, J. I. Sin, A. Tsai, L. Morrison, J. Oh, K. Dang, Y. Hu, K Kazahaya, M. Bennett, T. Dentchev, D. M. Wilson, A. A. Chalian, J. D. Boyer, M. G. Agadjanyan, D. B. Weiner. CD8 positive T cells controls antigen-specific immune responses through the expression of chemokines. J. Clin. Invest. 102, 1112-1124 (1998).
39. Cohen, J. AIDS Research: The mood is uncertain. Science 260,1254-1255 (1993).
40. Chao, B. H. et al. A 113-amino acid fragment of CD4 produced in Eschericha coli blocks human immunodefeciency virus-induced cell fusion. J. Biol Chem 264, 5812-5817 (1989).
41. Pantaleo, G., Menzo S, Vaccarezza M, Graziosi C, Cohen O Y, Demarest J F, Montefiori D, Orenstein J M, Fox C, Schrager L K, et. al. Studies in subjects with long-term nonprogressive Human immunodeficiency virus infection. New England Journal of Medicine 332, 209-216 (1995).
42. Berman, P., Gregory T J, Riddle L, Nakamura G R, Champe M A, Porter J P, Wurm F M, Hershberg R D, Cobb E K, Eichberg J W. Protection of chimpanzees from infection by HIV-1 after vaccination with recombinant glycoprotein gp120 but not gp160. Nature 345, 622-625 (1990).
43. Barrett, N., Eder G, Domer F. Characterization of a vaccinia-derived recombinant HIV-1 gp160 candidate vaccine and its immunogenicity in chimpanzees. Biotech Therapeut 2, 91-106 (1991).
44. Bruck, C., Thiriart C, Fabry L, et. al. HIV-1 envelope elicited neutralizing antibody titres correlate with protection and virus load in chimpanzees. Journal of Cellular Biochemistry 17, 88 (1993).
45. Girard, M., Kieny M P, Pinter A et. al. Immunization of chimpanzees confers protection against challenge with human immunodeficiency virus. Proc Natl Acad Sci USA 88, 542-546 (1991).
46. Cheng-Mayer, C., Seto D, Tateno M, Levy J A. Biologic features of HIV-1 that correlate with virulence in the host. Science 240, 80-82 (1988).
47. Katzenstein, D. A., Vujcic, L. K., Latif, A., Boulos, R., Halsey, N. A., Quinn, T. C., et. al. Human immunodeficiency neutralizing antibodies in sera from North Americans and Africans emergence of neutralization. J Acquir Immune Syndr 3, 810-816 (1990).
48. Pinto, L., Sullivan J, Berzofsky J A, Clerici M, Kessler H A, Landay A L, Shearer G M. Env-specific cytotoxic T lymphocyte responses in HIV seronegative health care workers occupationally exposed to HIV-contaminated body fluids. Journal of Clinical Investigation 96, 867-876 (1995).
49. Hulskotte, E. G., Geretti A-M, Siebelink K H, van Amerongen G, Cranage M P, Rud E W, Norley S G, de Vries P, Osterhaus A D. Vaccine-induced virus neutralizing antibodies and cytotoxic T cells do not protect macaques from experimental infection with Simian immunodeficiency virus SIV_{mac32H(J5)}. Journal of Virology 69, 6289-6296 (1995).
50. Clerici, M., Shearer G M. A Th1→Th2 switch is a critical step in the etiology of HIV infection. Immunology Today 14, 107-111 (1993).
51. Ruprecht, R. M., T. W. Baba, R. Rasmussen, Y. Hu, P. L. Sharma. Murine and simian retrovirus models: the threshold hypothesis. [Review]. AIDS 10 Suppl A, S33-40 (1996).
52. Wang, B., J. D. Boyer, K. E. Ugen, V. Srikantan, V. Ayyavoo, M. G. Agadjanyan, W. V. Williams, M. Newman, L. Coney, R. Carrano, D. B. Weiner. Nucleic acid-based immunization against HIV-1: induction of protective in vivo immune responses. AIDS 9, S159-170 (1995).
53. Kim, J. J., K. A. Simbiri, J. I. Sin, K. Dang, J. Oh, T. Dentchev, D. Lee, L. K. Nottingham, A. A. Chalian, M. G. Agadjanyan, and D. B. Weiner. Cytokine molecular adjuvants modulate immune responses induced by DNA vaccine constructs for HIV-1 and SIV. J. Interf. Cyto. Res. In Press.(1999).

54. Montefiori, D. C., W. E. Robinson, W. M. Mithell. Role of protein N-glycosylation in pathogenesis of human immunodeficiency virus type 1. Proc Natl Acad Sci USA 85, 9248-9252 (1988).

TABLE 1
Picornavirus Family

| | |
|---|---|
| Genera: | Rhinoviruses: (Medical) responsible for ˜50% cases of the common |
| | cold. |
| | Enteroviruses: (Medical) includes polioviruses, Coxsackieviruses, |
| | echoviruses, and human enteroviruses such as hepatitis A virus. |
| | Apthoviruses: (Veterinary) these are the foot and mouth disease |
| | viruses. |
| Target antigens: | VP1, VP2, VP3, VP4, VPG |

Calcivirus Family

| | |
|---|---|
| Genera: | Norwalk Group of Viruses: (Medical) these viruses are an important |
| | causative agent of epidemic gastroenteritis. |

Togavirus Family

| | |
|---|---|
| Genera: | Alphaviruses: (Medical and Veterinary) examples include Sindbis |
| | viruses, RossRiver virus and Eastern & Western Equine encephalitis. |
| | Rubivirus: (Medical) Rubella virus. |
| Flariviridue Family | Examples include: (Medical) dengue, yellow fever, Japanese |
| | encephalitis, St. Louis encephalitis and tick borne encephalitis |
| | viruses. |
| Hepatitis C Virus: | (Medical) these viruses are not placed in a family yet but are believed |
| | to be either a togavirus or a flavivirus. Most similarity is with |
| | togavirus family. |
| Coronavirus Family: | (Medical and Veterinary) |
| | Infectious bronchitis virus (poultry) |
| | Porcine transmissible gastroenteric virus (pig) |
| | Porcine hemagglutinating encephalomyelitis virus (pig) |
| | Feline infectious peritonitis virus (cats) |
| | Feline enteric coronavirus (cat) |
| | Canine coronavirus (dog) |
| | The human respiratory coronaviruses cause ˜40 cases of common |
| | cold. EX. 224E, 0C43 |
| | Note - coronaviruses may cause non-A, B or C hepatitis |
| Target antigens: | E1 - also called M or matrix protein |
| | E2 - also called S or Spike protein |
| | E3 - also called HE or hemagglutin-elterose glycoprotein (not |
| | present in all coronaviruses) |
| | N - nucleocapsid |

Rhabdovirus Family

| | |
|---|---|
| Genera: | Vesiculovirus: Vesicular Stomatitis Virus |
| | Lyssavirus: (medical and veterinary) rabies |
| Target antigens: | G protein |
| | N protein |
| Filoviridue Family: | (Medical) |
| | Hemorrhagic fever viruses such as Marburg and Ebola virus |

Paramyxovirus Family:

| | |
|---|---|
| Genera: | Parainfluenza Virus Type 1 |
| | Parainfluenza Virus Type 3 |
| | Bovine Parainfluenza Virus Type 3 |
| | Rubulavirus: (Medical and Veterinary) |
| | Mumps virus, Parainfluenza Virus Type 2, Parainfluenza Virus Type |
| | 4, NewCastle disease virus (important pathogen in chickens) |
| | Morbillivirus: (Medical and Veterinary) |
| | Measles, canine distemper |
| | Pneumonvirus: (Medical and Veterinary) |
| | Respiratory syncytial virus |
| Orthomyxovirus Family (Medical) | The Influenza virus |

Bunyavirus Family

| | |
|---|---|
| Genera: | Bunyavirus: (Medical) California encephalitis, La Crosse |
| | Phlebovirus: (Medical) Rift Valley Fever |
| | Hantavirus: Puremala is a hemahagin fever virus |
| | Nairovirus (Veterinary) Nairobi sheep disease |
| | Also many unassigned bungaviruses |
| Arenavirus Family (Medical) | LCM, Lassa fever virus |

Reovirus Family

| | |
|---|---|
| Genera: | Reovirus: a possible human pathogen |
| | Rotavirus: acute gastroenteritis in children |
| | Orbiviruses: (Medical and Veterinary) |
| | Cultivirus: Colorado Tick fever, Lebombo (humans) equine |
| | encephalosis, blue tongue |

Retrovirus Family

| | |
|---|---|
| Sub-Family: | Oncorivirinal: (Veterinary) (Medical) feline leukemia virus, HTLVI |
| | and HTLVII |
| | Lentivirinal: (Medical and Veterinary) HIV, feline |
| | immunodeficiency virus, equine infections, anemia virus |
| | Spumavirinal |

Papovavirus Family

| | |
|---|---|
| Sub-Family: | Polyomaviruses: (Medical) BKU and JCU viruses |
| Sub-Family: | Papillomavirus: (Medical) many viral types associated with cancers |
| | or malignant progression of papilloma |
| Adenovirus (Medical) | EX AD7, ARD., O.B. - cause respiratory disease - some adenoviruses such |
| | as 275 cause enteritis |
| Parvovirus Family (Veterinary) | Feline parvovirus: causes feline enteritis |
| | Feline panleucopeniavirus |
| | Canine parvovirus |
| | Porcine parvovirus |

Herpesvirus Family

| | |
|---|---|
| Sub-Family: | alphaherpesviridue |
| Genera: | Simplexvirus (Medical) |
| | HSVI, HSVII |
| | Varicellovirus: (Medical - Veterinary) pseudorabies - varicella zoster |
| Sub-Family - | betaherpesviridue |
| Genera: | Cytomegalovirus (Medical) |
| | HCMV |
| | Muromegalovirus |
| Sub-Family: | Gammaherpesviridue |
| Genera: | Lymphocryptovirus (Medical) |
| | EBV - (Burkitts lympho) |
| | Rhadinovirus |

Poxvirus Family

| | |
|---|---|
| Sub-Family: | Chordopoxviridue (Medical - Veterinary) |
| Genera: | Orthopoxvirus |
| | Variola (Smallpox) |
| | Vaccinia (Cowpox) |
| | Parapoxivirus - Veterinary |
| | Auipoxvirus - Veterinary |
| | Capripoxvirus |
| | Leporipoxvirus |
| | Suipoxvirus |
| Sub-Family: | Entemopoxviridue |
| Hepadnavirus Family: | Hepatitis B virus |
| Unclassified: | Hepatitis delta virus |

TABLE 2
Bacterial pathogens

Pathogenic gram-positive cocci include: pneumococcal; staphylococcal; and
streptococcal. Pathogenic gram-negative cocci include: meningococcal; and gonococcal.
Pathogenic enteric gram-negative bacilli include: enterobacteriaceae; pseudomonas,
acinetobacteria and eikenella; melioidosis; salmonella; shigellosis; hemophilus;
moraxella; chancroid; brucellosis; tularemia; yersinia (pasteurella); streptobacillus
moniliformis and spirillum; listeria monocytogenes; erysipelothrix rhusiopathiae;
diphtheria; cholera; anthrax; donovanosis (granuloma inguinale); and bartonellosis.
Pathogenic anaerobic bacteria include: tetanus; botulism; other clostridia;
tuberculosis; leprosy; and other mycobacteria. Pathogenic spirochetal diseases
include: syphilis; treponematoses: yaws, pinta and endemic syphilis; and
leptospirosis.
Other infections caused by higher pathogen bacteria and pathogenic fungi include:
actinomycosis; nocardiosis; cryptococcosis, blastomycosis, histoplasmosis and
coccidioidomycosis; candidiasis, aspergillosis, and mucormycosis; sporotrichosis;
paracoccidiodomycosis, petriellidiosis, torulopsosis, mycetoma and chromomycosis;
and dermatophytosis.
Rickettsial infections include rickettsial and rickettsioses.
Examples of mycoplasma and chlamydial infections include: mycoplasma
pneumoniae; lymphogranuloma venereum; psittacosis; and perinatal chlamydial
infections.

Pathogenic eukaryotes

Pathogenic protozoans and helminths and infections thereby include: amebiasis;
malaria; leishmaniasis; trypanosomiasis; toxoplasmosis; pneumocystis carinii;
babesiosis; giardiasis; trichinosis; filariasis; schistosomiasis; nematodes; trematodes
or flukes; and cestode (tapeworm) infections.

1. A method of introducing a compound into a cell that expresses costimulatory molecules, said method comprising contacting the cell with a non-cellular particle that comprises the compound and a costimulatory ligand.

2. The method of claim 1 wherein the compound is a nucleic acid molecule or protein.

3. The method of claim 1 wherein the compound is DNA.

4. The method of claim 1 wherein the compound is DNA that comprises a nucleotide sequences that encodes a protein operably linked to regulatory elements functional in the cell.

5. The method of claim 1 wherein the compound is DNA that comprises a nucleotide sequences that encodes an immunogenic protein operably linked to regulatory elements functional in the cell.

6. The method of claim 1 wherein the compound is DNA that comprises a nucleotide sequences that encodes an non-immunogenic protein operably linked to regulatory elements functional in the cell.

7. The method of claim 1 wherein the compound is a viral protein.

8. The method of claim 1 wherein the cell that expresses costimulatory molecules is a dendretic cell or a macrophage cell

9. The method of claim 1 wherein the costimulatory ligand is an antibody or a native ligand of a costimulatory molecule.

10. The method of claim 1 wherein the costimulatory ligand is a fusion protein that includes a costimulatory ligand portion and a viral protein portion.

11. The method of claim 1 wherein the particle is a viral particle, a protein complex, a liposome or a cationic amphiphile/DNA complex.

12. A method of introducing a compound into a cell comprising contacting the cell with a particle that comprises the compound and a fusion protein, the fusion protein comprising the extracellular region of CD28 and the transmembrane and cytoplasmic regions of HIV-1 gp41.

13. A method of delivering a therapeutic protein an individual comprising the step of administering to tissue of said individual at a site on said individual's body, a particle that comprises therapeutic protein or a nucleic acid molecule that encodes a therapeutic protein, and costimulatory ligand.

14. The method of claim 13 wherein the particle contains a non-immunogenic therapeutic protein or a DNA molecule that encodes an non-immunogenic therapeutic protein.

15. The method of claim 13 wherein the particle contains a growth factor or cytokine or a DNA molecule that encodes a growth factor or cytokine.

16. The method of claim 13 wherein the particle is a viral particle, a protein complex, a liposome or a cationic amphiphile/DNA complex.

17. A method of immunizing against cancer comprising administering to an individual, a cancer cell comprising a recombinant expression vector that encodes a costimulatory ligand.

18. A particle that comprises a compound and a costimulatory ligand.

19. The particle of claim 18 which the costimulatory ligand is a fusion protein comprising the extracellular region of CD28 and the transmembrane and cytoplasmic regions of HIV-1 gp41.

20. The particle of claim 18 wherein the compound is an nucleic acid or protein.

21. The particle of claim 18 wherein the compound is DNA.

22. The particle of claim 18 wherein the compound is DNA that comprises a nucleotide sequences that encodes an immunogenic protein operably linked to regulatory elements functional in the cell.

23. The particle of claim 18 wherein the compound is DNA that comprises a nucleotide sequences that encodes an non-immunogenic protein operably linked to regulatory elements functional in the cell.

24. The particle of claim 18 wherein the particle is a viral particle, a protein complex, a liposome or a cationic amphiphile/DNA complex.

25. A cancer cell comprising a recombinant expression vector that encodes a costimulatory ligand.

26. A method of immunizing an individual comprising the steps of administering to tissue of said individual at a site on said individual's body, a DNA molecule that comprises a nucleotide sequence that encodes an immunogenic protein operably linked to regulatory elements, subsequently administering to said individual a particle that comprises an immunogenic protein.

27. The method of claim 26 wherein said particle further comprises a compound.

28. The method of claim 27 wherein the compound is a nucleic acid molecule.

29. The method of claim 28 wherein the compound is DNA.

30. The method of claim 29 wherein the compound is DNA that comprises a nucleotide sequences that encodes an immunogenic protein operably linked to regulatory elements functional in the cell.

31. The method of claim 26 wherein the particle is a viral particle or a protein complex.
